(19) United States
(12) Patent Application Publication
(10) Pub. No.: US 2023/0348917 A1
(43) Pub. Date: Nov. 2, 2023

(54) EXPRESSION VECTOR FOR USE IN METHANOTROPH

(71) Applicant: KOREA RESEARCH INSTITUTE OF BIOSCIENCE AND BIOTECHNOLOGY, Daejeon (KR)

(72) Inventors: Seung Goo LEE, Daejeon (UA); Dae Hee Lee, Daejeon (KR); Hye Won Lee, Daejeon (KR); Seong Keun Kim, Daejeon (KR); Ji In Baek, Daejeon (KR); Tae Hyun Kim, Daejeon (KR); So Hyung Oh, Daejeon (KR)

(73) Assignee: KOREA RESEARCH INSTITUTE OF BIOSCIENCE AND BIOTECHNOLOGY, Daejeon (KR)

(21) Appl. No.: 18/189,783

(22) Filed: Mar. 24, 2023

Related U.S. Application Data
(63) Continuation of application No. PCT/KR21/13043, filed on Sep. 24, 2021.

(30) Foreign Application Priority Data
Sep. 25, 2020 (KR) 10-2020-0125080
Sep. 17, 2021 (KR) 10-2021-0124777

(51) Int. Cl. C12N 15/74; C12N 1/20

(52) U.S. Cl. CPC C12N 15/74; C12N 1/205; C12N 2800/101; C12R 2001/01

(57) ABSTRACT

Disclosed are a vector comprising a first expression cassette comprising a DmpR ribosomal binding site as a transcriptional regulator and a promoter operably linked thereto; and a second expression cassette comprising a sequence encoding a target polypeptide, a ribosomal binding site, and a Po promoter operably linked thereto, and a methanotroph transformed by the vector.

### CROSS REFERENCE TO RELATED APPLICATION

The present application claims priority to Korean Patent Application Nos. 10-2020-0125080, filed Sep. 25, 2020 and 10-2021-0124777, filed Sep. 17, 2021, the entire contents of which is incorporated herein for all purposes by this reference.

### BACKGROUND OF THE INVENTION

### Field of the Invention

The disclosure relates to expression vectors for use in methanotrophs.

### Description of the Related Art

Methane, which is the main component of natural gas, shale gas and biogas, is a global warming gas and is attracting attention as a next-generation carbon resource as well as a target for reduction. A conventional chemical methane conversion method is a multi-step process, and has low reaction efficiency, and there are problems due to process operation conditions of high temperature/high pressure.

As an alternative to overcome these disadvantages, a biological conversion method has recently been developed. Methanotrophs grow using, as a carbon source, methane at room temperature and under normal pressure using methane monooxygenase. Therefore, it is theoretically possible to produce various kinds of high value-added products from methane based on the metabolic pathway of methanotrophs.

However, despite this possibility, in the case of transforming methanotrophs using previously known molecular biological tools (e.g., vectors, expression cassettes, and promoters) for gene expression, metabolism control, or the like, expected effects are often not achieved. Therefore, methanotrophs are not actively utilized industrially. In particular, existing methanotrophs expression systems mainly depend on a constitutive promoter, and in the methanotrophs transformed with an expression system having an inducible promoter, there is a problem in that the expression of the target protein is uneven between cells and the expression level is not sufficient.

A CRISPR/Cas system is used for genome editing of various organisms, but it has not been actively used in methanotrophs for which a tight-regulated expression control system for precise expression regulation of toxic Cas protein has not been reported. In methanotrophs, genome is edited mainly by homologous recombination, but when the homologous recombination is used, the efficiency is low, so the editing depends on a two-step process of 1) introducing a mutant gene with an antibiotic marker, 2) selecting it and removing the antibiotic marker. The genome editing method through this two-step process was labor-intensive and time-consuming, especially when several mutations were sequentially introduced into slow-growing methanotrophs. Therefore, a powerful inducible expression system is essential for the development of efficient genome engineering tools using the CRISPR/Cas system in methanotrophs.

### SUMMARY OF THE INVENTION

One embodiment provides a vector that can be used for expressing a target polypeptide in methanotrophs, regulating or promoting the expression of target genes of methanotrophs to reach a desired expression level, or for other genetic manipulations of methanotrophs.

One aspect of the disclosure provides a vector for introduction into a methanotroph, comprising a first expression cassette comprising DmpR as a transcriptional regulator and a promoter operably linked thereto; and a second expression cassette comprising a sequence encoding a target polypeptide and a Po promoter operably linked thereto.

In one embodiment, the target polypeptide may be a marker, a reporter, a polypeptide capable of improving production of a desired metabolite, or a polypeptide capable of promoting growth of the methanotroph.

In one embodiment, in the vector, expression of the target polypeptide may be induced by a phenolic compound.

In one embodiment, transcription directions of the first expression cassette and the second expression cassette may be the same or opposite.

In one embodiment, the vector may be a plasmid.

In one embodiment, the methanotroph may be selected from the group consisting of Methylomonas sp., Methylobacter sp., Methylococcus sp., Methylosphaera sp., Methylocaldum sp., Methyloglobus sp., Methylosarcina sp., Methyloprofundus sp., Methylothermus sp., Methylohalobius sp., Methylogaea sp., Methylomarinum sp., Methylovulum sp., Methylomarinovum sp., Methylorubrum sp., Methyloparacoccus sp., Methylosinus sp., Methylocystis sp., Methylocella sp., Methylocapsa sp., Methylofurula sp., Methylacidiphilum sp., Methylacidimicrobium sp., and Methylomicrobium sp.

In one embodiment, the methanotroph may be Methylococcus capsulatus Bath or Methylosinus trichosporium OB3b.

Another aspect of the disclosure provides a methanotroph in which the above vector is introduced.

The expression vector according to one embodiment can precisely regulate the expression time and amount of a specific gene in methanotrophs by inducible expression.

Methanotrophs into which the expression vector according to one embodiment is introduced show a uniform expression level of individual cells, so research can be conducted using various high-speed mass-analysis techniques that can be observed at the individual cell level, such as flow cytometry and microscopy, thereby the speed and efficiency of methanotrophs research.

Since the methanotrophs into which the expression vector according to one embodiment is introduced show excellent expression of the target protein, they can be used for effective mass production of recombinant proteins using methane gas.

The expression vector according to one embodiment can be used for metabolic engineering or synthetic biology of methanotrophs.

The vector according to one embodiment can be used to improve the efficiency of transformation and metabolic manipulation of methanotrophs, regulate target gene expression, or increase target gene expression.

### BRIEF DESCRIPTION OF THE DRAWINGS

FIG. 1 shows a vector map of a pAWP89 plasmid.

FIG. 2 shows an expression cassette and its inducers according to an embodiment.

FIG. 3 shows a result of confirming an expression level of dTomato in M. trichosporium OB3b into which a vector containing a TetR-Pₜₑₜ-dTomato expression cassette is introduced by a concentration of an anhydrotetracycline (aTc) inducer and over time. (A) shows a measurement of an average fluorescence value/OD₆₀₀ nm using a microplate reader, and (B) shows a distribution by observing fluorescence expression of individual cells with a flow cytometer.

FIG. 4 shows a result of confirming an expression level of dTomato in M. capsulatus Bath into which a vector containing a DmpR-Pₒ-dTomato, LacI-P_{trc}-dTomato, or XylS-Pₘ-dTomato expression cassette is introduced by a concentration of an inducer by measuring an average fluorescence value/OD₆₀₀ₙₘ.

FIG. 5 shows a result of confirming an expression level distribution of dTomato in M. capsulatus Bath into which a vector containing a LacI-P_{trc}-dTomato, XylS-Pₘ-dTomato or DmpR-Pₒ-dTomato expression cassette is introduced by a concentration of an inducer by measuring fluorescence intensities of individual cells.

FIG. 6 is a result of confirming an expression level of dTomato in M. capsulatus Bath into which a vector containing a DmpR-Pₒ-dTomato expression cassette is introduced by SDS-PAGE.

FIG. 7 is a result of confirming a difference in dTomato expression levels between wild-type M. capsulatus Bath and M. capsulatus Bath in which a vector containing a DmpR-Pₒ-dTomato expression cassette is introduced in the presence of 10 μM phenol and in the absence of phenol.

FIG. 8 is a result of confirming dTomato expression level of M. capsulatus Bath in which a vector containing a DmpR-Pₒ-dTomato expression cassette is introduced over time under phenol condition of 10 μM concentration.

FIG. 9 shows a result of confirming an expression level of dTomato in M. trichosporium OB3b into which a vector containing a DmpR-Pₒ-dTomato, LacI-P_{trc}-dTomato, or XylS-Pₘ-dTomato expression cassette is introduced by inducer concentration by measuring an average fluorescence value/OD₆₀₀ₙₘ.

FIG. 10 shows a result of confirming an expression level distribution of dTomato in M. trichosporium OB3b into which a vector containing a LacI-P_{trc}-dTomato, XylS-Pₘ-dTomato or DmpR-Pₒ-dTomato expression cassette is introduced by inducer concentration by measuring fluorescence intensities of individual cells.

FIG. 11 shows a result of confirming an expression level of dTomato in M. trichosporium OB3b in which a vector containing a DmpR-Pₒ-dTomato expression cassette is introduced by SDS-PAGE.

FIG. 12 is a result of confirming whether dTomato is induced under sMMO expression condition or pMMO expression condition by supplying benzene to M. capsulatus Bath into which a vector containing a DmpR-Pₒ-dTomato expression cassette is introduced.

FIG. 13 is a diagram showing improvement of an expression system for methanotrophs according to a ribosome binding site.

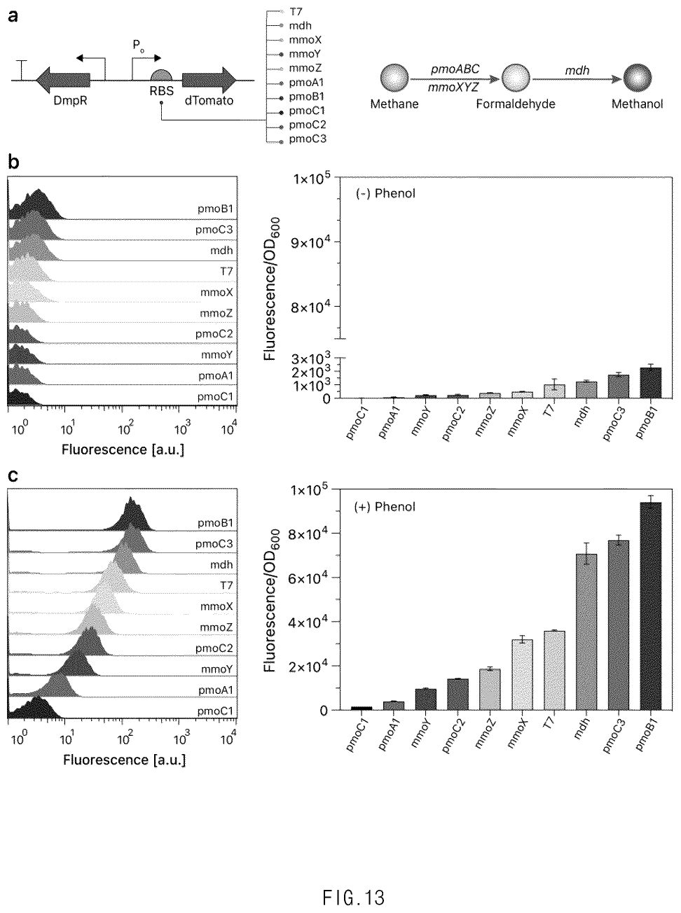

FIG. 14 is a result of confirming gene expression according to the presence or absence of phenol in M. capsulatus Bath containing a pAWP78 vector, a FmDTA-pAWP or Po-FmDTA-pAWP expression cassette.

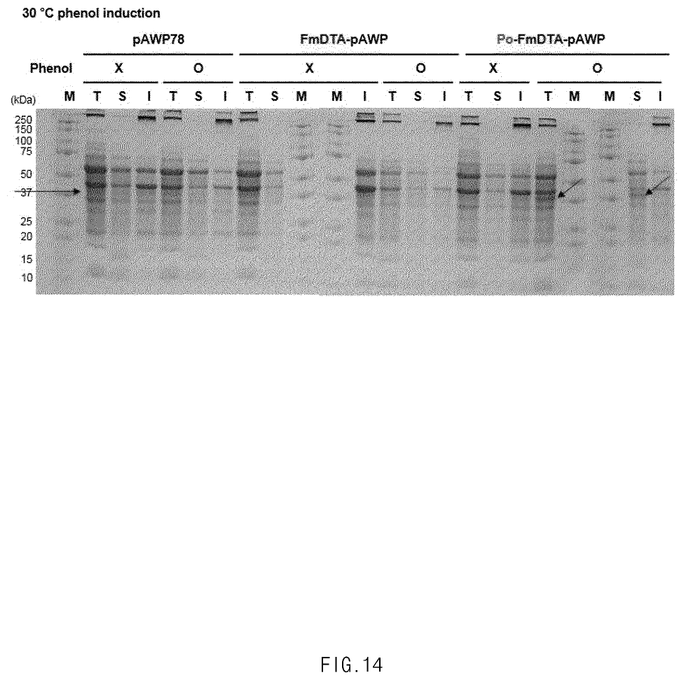

FIG. 15 is a diagram showing a construction of a CRISPR/Cas9-based cytosine base editor plasmid and a conversion rate of point mutations from cytosine to thymine using the constructed plasmid.

FIG. 16 is a diagram showing isoprene production in M. capsulatus Bath. (a) shows an isoprene biosynthetic pathway in M. capsulatus Bath, (b) shows a plasmid construct used to produce isoprene through a mevalonate pathway, and (c) shows a fermentation profile of a strain containing a plasmid.

FIG. 17 is a diagram of the toxicity of isoprene and epoxy isoprene. (a) shows that isoprene oxidation by sMMO causes cytotoxicity due to the formation of epoxy isoprene, whereas pMMO shows no activity for isoprene and therefore no formation of toxic products. (b, c) show growth profiles over time of sMMO and pMMO expressing cells controlled by addition or no addition of copper in a medium in the presence of different concentrations of isoprene. (d, e) shows growth profiles over time of sMMO and pMMO expressing cells in the presence of different concentrations of epoxy isoprene.

FIG. 18 shows a result of evaluating efficacy by inserting a stop codon into a mmoX gene (sMMO subunit) using a high-efficiency CRISPR/Cas9-based cytosine-based editor.

FIG. 19 shows a result of high-density cell fermentation of wild type ((a) Pₒ-PtIspS-MVA/Bath) and ΔmmoX mutant strain ((b) Pₒ-PtIspS-MVA/ΔmmoX).

### DETAILED DESCRIPTION OF THE INVENTION

One aspect provides a vector for introduction into a methanotroph, comprising a first expression cassette containing DmpR as a transcriptional regulator and a promoter operably linked thereto; and a second expression cassette containing a sequence encoding a target polypeptide and a Po promoter operably linked thereto.

The vector may be used for artificially manipulating the metabolic process of methanotrophs such as heterologous expression in methanotrophs according to the type of sequence encoding a target polypeptide contained therein, or regulation or promotion of the production of endogenous proteins. The vector may be for artificially regulating the metabolic process of methanotrophs or constructing a new metabolic circuit. The vector may be for increasing or decreasing the productivity of an endogenous substance. The vector may be for imparting new substance production capability. The vector may be for increasing or controlling the growth rate of methanotrophs. The vector may be for engineering methanotrophs. The term “engineering” means introducing a vector containing a polynucleotide encoding a target polypeptide into a host cell so that a protein encoded by the polynucleotide can be expressed in the host cell.

The term “operably linked” means that DNA regions are functionally related to each other. For example, In the case that a promoter regulates transcription of a gene sequence, a promoter and a gene sequence are operably linked.

The methanotrophs are strains that can grow using methane as a carbon source at room temperature and under normal pressure using methane monooxygenase (MMO). The methane monooxygenase is classified into soluble methane monooxygenase (sMMO) expressed in the cytoplasm and particulate methane monooxygenase (pMMO) expressed in the cell membrane. The MMO oxidizes methane to methanol, and methanol is converted to formaldehyde and then enters the serine pathway and/or RuMP pathway depending on the type of methanotrophs.

The di-methyl phenol regulator (DmpR) is a transcription factor for the phenol degradation, and may be derived from a microorganism of Pseudomonas sp. The DmpR exists as an unreactive dimer, but when combined with phenol, it changes to a polymer such as a tetramer or hexamer in which four molecules are bonded. The DmpR polymers formed by the reaction with phenol may bind to a Po promoter and express a target polypeptide operably linked to the Po promoter. According to one embodiment, the expression of dTomato in methanotrophs in which a vector containing DmpR is introduced is higher than that in methanotrophs in which a vector containing TetR, LacI, or XylS is contained as a regulator.

The sequence of the DmpR gene may consist of the nucleotide sequence of SEQ ID NO: 1. The DmpR, which consists of the above SEQ ID NO: 1, may include a polynucleotide including nucleotide sequence that has at least 60% or more, 70% or more, 80% or more, 83% or more, 84% or more, 88% or more, 90% or more, 93% or more, 95% or more, or 97% or more homology to the nucleotide sequence of SEQ ID NO: 1. Any polynucleotide sequence with deletion, modification, substitution, or addition in part of the sequence may also be included within the scope of the disclosure, as long as the sequence has the homology to the above sequence and exhibits biological activity substantially identical to or corresponding to that of the above sequence.

As the transcriptional regulator, a gene encoding a DmpR variant may be used instead of wild-type DmpR. The DmpR variant may be a variant with improved phenol sensitivity and substrate specificity. For example, the variant may be one in which the 71st glutamine (Q) is substituted with alanine (A), the 135th glutamic acid (E) is substituted with lysine (K), or the 188th lysine (K) is substituted with arginine (R) in the wild-type DmpR consisting of the amino acid sequence of SEQ ID NO: 35. Specifically, the DmpR variant may include one or more mutations selected from the group consisting of M52I, M52C, Q71A, E135K, and K188R. For example, the DmpR variant may be DmpR(M52I), DmpR(M52C), DmpR(M52I/K188R), DmpR(M52I/Q71A) or DmpR(M52C/E135K). When the gene encoding the DmpR variant is introduced as a transcriptional regulator, phenol sensitivity and substrate specificity can be improved compared to using the wild-type DmpR gene.

The DmpR variant may be a DmpR variant capable of inducing constitutive expression by forming polymer even in the absence of phenol. Since this can activate expression by the Po promoter, constitutive expression of the target polypeptide can also be achieved. The DmpR variant inducing constitutive expression may consist of the DmpR (Δ2-204) sequence of SEQ ID NO: 2, which is a sequence encoding DmpR (Δ2-204) in which 2^{nd} to 204^{th} amino acids of the wide DmpR are deleted.

The term “homology” may indicate the degree of matching with the given nucleotide sequence, and may be presented as a percentage (%). In the specification, a homology sequence having an activity which is identical or similar to the given nucleotide sequence is presented as “% homology”. The homology to the nucleotide sequence can be determined by, for example, algorithm BLAST (see Karlin and Altschul, Pro. Natl. Acad. Sci. USA, 90, 5873, 1993) or FASTA (see Pearson, Methods Enzymol., 183, 63, 1990). Based on this algorithm BLAST, programs called BLASTN or BLASTX have been developed (see http://www.ncib.nlm.nth.gov).

The Po promoter can activate the transcription of a target polypeptide operably linked thereto by binding to the multimerized DmpR in reaction with phenol. Information on the Po promoter may be found in ACS Synth Biol. 2014 Mar. 21; 3(3):163-171. The sequence of the Po promoter may consist of the nucleotide sequence of SEQ ID NO: 3. The Po promoter, which consists of SEQ ID NO: 2, may include a polynucleotide including nucleotide sequence that has at least 60% or more, 70% or more, 80% or more, 83% or more, 84% or more, 88% or more, 90% or more, 93% or more, 95% or more, or 97% or more homology to the sequence of SEQ ID NO: 2. Any polynucleotide sequence with deletion, modification, substitution, or addition in part of the sequence may also be included within the scope of the disclosure, as long as the sequence has the homology to the above sequence and exhibits biological activity substantially identical to or corresponding to that of the above sequence.

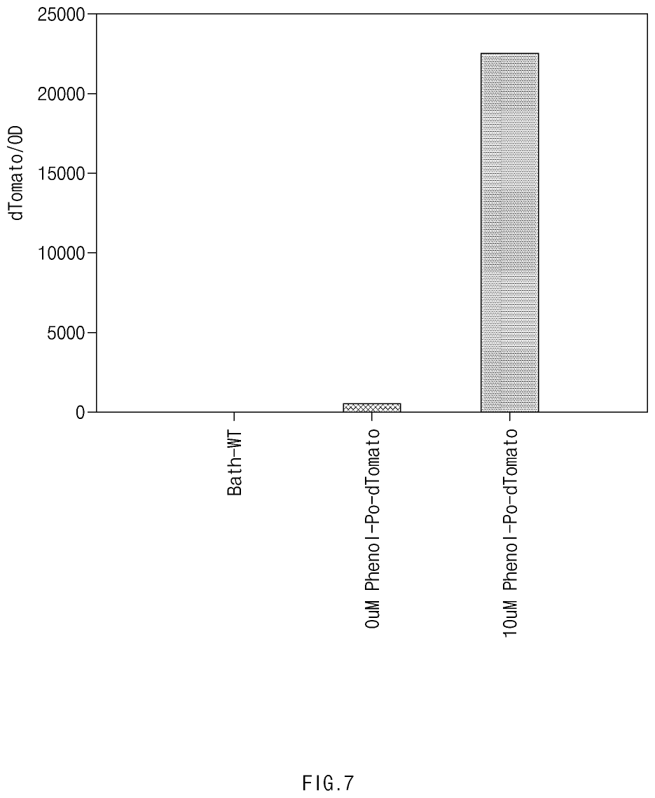

Since the vector can tightly regulate the inducible expression of the target polypeptide, there is an advantage in that there is no or very low leaky expression of the target polypeptide in the absence of an inducer. The leaky expression means a basal level expression that occurs in the absence of an inducer. According to FIG. 7, assuming that the vector has 100% fluorescence by dTomato expression in the presence of 10 μM phenol, the rate of fluorescence by dTomato expression in the absence of phenol is maintained at a very low level of 2% or less, resulting in excellent inhibition efficacy of leak expression.

The vector may further include a ribosomal binding site (RBS). The ribosomal binding site may also be referred to as a Shine-Dalgarno sequence. The ribosomal binding site may be located upstream of a target protein gene sequence or between a promoter gene sequence and a target protein sequence. A spacer sequence may be further included between the ribosomal binding site and the initiation codon of the target protein. The sequence of the ribosomal binding site may be a methanotroph-derived RBS sequence.

The sequence of the ribosomal binding site may consist of the nucleotide sequence of SEQ ID NO: 4. The nucleotide sequence of SEQ ID NO: 4 is a Bacteriophage T7 gene 10-derived sequence.

The ribosomal binding site sequence may be selected from the group consisting of the RBS sequence of mxaF(MDH) of SEQ ID NO: 5, the RBS sequence of mmoX of SEQ ID NO: 6, the RBS sequence of mmoY of SEQ ID NO: 7, the RBS sequence of mmoZ of SEQ ID NO: 8, the RBS sequence of pmoA1 of SEQ ID NO: 9, the RBS sequence of pmoB1 of SEQ ID NO: 10, the RBS sequence of pmoC1 of SEQ ID NO: 11, the RBS sequence of pmoC2 of SEQ ID NO: 12, and the RBS sequence of pmoC3 of SEQ ID NO: 13. A vector containing the ribosomal binding site sequence consisting of a sequence selected from the sequences of SEQ ID NOs: 6 to 13 can increase the expression of a target polypeptide in methanotrophs than that in a vector containing the RBS sequence of methanol dehydrogenase (MDH).

The RBS, which consists of the sequences of the above SEQ ID NOs: 4 to 13, may include a polynucleotide including nucleotide sequence that has at least 60% or more, 70% or more, 80% or more, 83% or more, 84% or more, 88% or more, 90% or more, 93% or more, 95% or more, or 97% or more homology to the respective nucleotide sequences. Any polynucleotide sequence with deletion, modification, substitution, or addition in part of the sequence may also be included within the scope of the disclosure, as long as the sequence has the homology to the above sequences and exhibits biological activity substantially identical to or corresponding to that of the above sequences.

The target polypeptide may be, for example, a marker or reporter. The target polypeptide may be for the production of a foreign protein. The target polypeptide may be for improving the production of a desired metabolite, promoting the growth of methanotrophs, producing a specific protein, changing or inhibiting the production of metabolites of methanotrophs, or inhibiting the expression or activity of a specific enzyme. The target polypeptide may be, for example, formate dehydrogenase (FDH), particulate methane monooxygenase (pMMO), threonine aldolase (TA), isoprene synthase, or lactate dehydrogenase, but is not limited thereto. The marker or reporter protein may be a fluorescent protein or an antibiotic resistance gene protein, for example, a fluorescent protein such as dTomato, but is not particularly limited. The dTomato may consist of the nucleotide sequence of SEQ ID NO: 14.

The sequence encoding the target polypeptide may be a codon-optimized sequence for the methanotroph to be introduced.

The vector may contain multiple cloning sites for introducing the sequence encoding the target polypeptide, in place of the sequence. The multiple cloning sites are sites into which genes encoding a target polypeptide or target protein to be expressed through the expression vector of the disclosure are inserted.

According to one embodiment, the vector may induce the expression of the target polypeptide by a phenolic compound. Since sMMO of methanotrophs may oxidize benzene to phenol, the vector may activate the expression of the target protein even by benzene. According to one embodiment, the present inventors confirmed that phenol was superior to other expression inducers such as aTc and IPTG in inducing the expression of methanotrophs. Compared to aTc, IPTG, and benzoate, phenol has a relatively small molecular weight and size, and is more hydrophilic or amphipathic, so it may be easy to activate the expression of the inducible vector inside the strain by penetrating, absorbing, or being received by methanotrophs. In addition, there is an advantage in that homogenous expression can be achieved because there is little difference in expression level between methanotroph cells. According to one embodiment, methanotrophs that induced expression by phenol showed significantly higher dTomato expression and a low and uniform intercellular distribution despite the use of a lower amount of phenol, compared to those that induced expression by anhydrotetracycline (aTc), IPTG, and benzoate. This may be due to the difference in the receptibility of methanotrophs to the inducer. The phenolic compound may include phenol, 2-chlorophenol, 2-iodophenol, o-cresol, 2-ethylphenol, m-cresol, 2-nitrophenol, catechol, 2-methoxyphenol, 2-aminophenol, 2,3-dichlorophenol, 3-chlorophenol, 2,3-dimethylphenol, 3-nitrophenol, 4-chlorophenol, p-cresol, 2,5-dichlorophenol, 2,5-dimethylphenol, or salicylic acid.

The expression cassette refers to a region that includes a coding sequence of a specific gene and a sequence regulating its expression. For example, the expression cassette may include a polynucleotide encoding a target polypeptide, a promoter operably linked thereto, a transcription termination signal, a ribosomal binding site, and a translation termination signal.

According to one embodiment, the transcription directions of the first expression cassette and second expression cassette may be the same or opposite. Reversely aligning the transcriptional directions may be advantageous for precise regulation of expression levels.

The vector may be a plasmid. Specifically, the plasmid may be a plasmid capable of delivery by conjugation to methanotrophs and replication in methanotrophs. For example, the plasmid may be a pBR-based, pUC-based, pBluescriptII-based, pGEM-based, pTZ-based, pCL-based or pET-based plasmid. Specifically, the plasmid may include pDZ, pACYC177, pACYC184, pCL, pECCG117, pUC19, pBR322, pMW118, pCC1BAC, pUC18, pBAD, pJK001, pCM, or pAWP plasmid, preferably pAWP, but is not particularly limited. According to one embodiment, the vector of the disclosure may be constructed by introducing the DmpR and the Po promoter into the pAWP89 vector. The pAWP89 vector may consist of the sequence of SEQ ID NO: 15. The pAWP89 plasmid may be prepared or purchased commercially, and for example, the one from addgene's catalog #61264 may be used.

The methanotrophs may be selected from the group consisting of Methylomonas sp., Methylobacter sp., Methylococcus sp., Methylosphaera sp., Methylocaldum sp., Methyloglobus sp., Methylosarcina sp., Methyloprofundus sp., Methylothermus sp., Methylohalobius sp., Methylogaea sp., Methylomarinum sp., Methylovulum sp., Methylomarinovum sp., Methylorubrum sp., Methyloparacoccus sp., Methylosinus sp., Methylocystis sp., Methylocella sp., Methylocapsa sp., Methylofurula sp., Methylacidiphilum sp., Methylacidimicrobium sp., and Methylomicrobium sp., and more specifically, Methylococcus sp. or Methylosinus sp.

The methanotrophs may be gamma-proteobacteria (also referred to as Group I), alpha-proteobacteria (also referred to as Group II), or verrucomicrobia (also referred to as Group III). Among them, gamma-proteobacteria are strains that use the RuMP pathway as a main metabolic pathway, and alpha-proteobacteria are strains that use the serine pathway as a main metabolic pathway.

The methanotrophs may be Methylococcus capsulatus Bath or Methylosinus trichosporium OB3b.

The expression vector may be an inducible expression vector. An inducible expression vector is introduced into methanotrophs, and is cultured until a sufficient number of strains are secured. Then, the target polypeptide may be expressed by adding an inducer in the culture. This has the advantage of increasing the efficiency of the strain growth and the target protein production because it can induce the expression of a specific gene at a necessary time compared to the case of using a constitutive expression vector.

The vector may include a replication origin or an origin of replication (oriV) that functions in methanotrophs. The vector may further include an origin of replication that functions in bacteria other than methanotrophs. The non-methanogenic bacteria may be donor bacteria of conjugation. The oriV may be, for example, oriV_{RP4/RK2} and/or oriV_{ColE1}. The oriV_{RP4/RK2} may consist of the sequence of SEQ ID NO: 16. The oriV_{ColE1} may consist of the sequence of SEQ ID NO: 17.

The vector may include an origin of transfer (oriT) for conjugative transfer, and may include, for example, oriT_{RP4/RK2}. The oriT may consist of the sequence of SEQ ID NO: 18.

The vector may include a sequence encoding a protein activating the origin of replication or a replication initiation protein, for example, a sequence encoding a TrfA protein. The TrfA may consist of the sequence of SEQ ID NO: 19.

The vector may include a sequence for plasmid maintenance and conjugative transfer, and may include, for example, a traJ or traJ′ gene sequence. The traJ may consist of the sequence of SEQ ID NO: 20. The traJ′ may consist of the sequence of SEQ ID NO: 21.

The vector may include an antibiotic resistance gene, for example, a kanamycin resistance gene (KanR). The KanR may consist of the sequence of SEQ ID NO: 22.

Another aspect provides a methanotroph into which the above vector is introduced. The methanotroph introduced with the vector may express a heterologous polypeptide by expressing the target polypeptide of the vector, or express an endogenous polypeptide. Alternatively, as the amount or pathway of metabolism is regulated, the expression of a specific gene may be increased or inhibited, or the growth of methanotrophs may be promoted or inhibited.

The methanotroph that may be used for the introduction is the same as that described above.

The vector may be introduced by conjugation, heat shock, or electroporation. The conjugation is mainly used to transfer nucleic acids to methanotrophs. The conjugation involves mixing donor cells and recipient cells. A donor cell may be a cell containing the vector. The conjugation may be performed by further mixing helper cells. The conjugation may be a biparental mating conjugation or a triparental mating conjugation. A vector delivery method by conjugation of methanotrophs is known to those skilled in the art, and for example, reference may be made to information disclosed in Stolyar et al., 1995, Mikrobiologiya 64: 686-691.

Another aspect provides a method for producing a target substance including culturing the vector-introduced methanotroph; and collecting a target substance from the culture of the methanotroph.

The target substance may include the methanotroph itself, single cell protein, biopolymers produced by methanotrophs such as methanol, formaldehyde, PHA or PHB, or a heterologous protein produced by the expression of the target polypeptide contained in the vector. The methanotroph in which the vector is introduced may produce new proteins by the genes contained in the vector, and the expression of endogenous genes may be increased or decreased by regulating or promoting metabolism, or the growth of the methanotroph may be promoted or inhibited.

The culture may be performed by a batch culture method, a continuous culture method, or a fed-batch culture method, and specifically, it may be cultured continuously in a fed batch or repeated fed batch process.

The culture may be cultured in a liquid medium or a solid medium. The culture may use an NMS medium known to be used for culturing methanotrophs. The medium may include a C1 substrate as a carbon source. The C1 substrate refers to a molecule without a bond between carbon and carbon, and may be, for example, methane, methanol, formate, formaldehyde, methylated amine, methylthiol, or carbon monoxide. The C1 substrate may be cultured by supplying, for example, 10, 20, 30, 40, 50, 60, or 70% methane gas based on a headspace. The medium may be a copper-deficient or copper-added medium for expressing sMMO or pMMO in methanotrophs, and specifically, CuCl₂ may be added.

The culture may be agitated at 150 rpm to 300 rpm, 180 rpm to 270 rpm, or 200 rpm to 250 rpm to facilitate methane transport. The incubation temperature may be 15 to 45° C., 20 to 40° C., or 25 to 35° C., and may be appropriately adjusted according to the type of methanotrophs.

The production method may further include expressing the target polypeptide by adding an inducer to the methanotroph culture. An inducer for inducing expression of the target polypeptide may be added to the medium. The inducer may be phenol or a phenolic compound. For example, the medium may contain 1 to 100 μM, 5 to 10 μM, or 10 μM of phenol.

The obtained product may be collected by a method of centrifugation, filtration, extraction, spraying, drying, evaporation, precipitation, crystallization, electrophoresis, fractional dissolution (e.g., ammonium sulfate precipitation), chromatography (e.g., HPLC, ion exchange, affinity, hydrophobic, and size exclusion), or the like.

### MODE FOR THE INVENTION

Hereinafter, one or more specific embodiments will be described in more detail through examples. However, these examples are intended to illustrate one or more specific embodiments, and the scope of the disclosure is not limited to these examples.

### Experimental Example 1: Production of Inducible Expression Plasmid Expressing dTomato

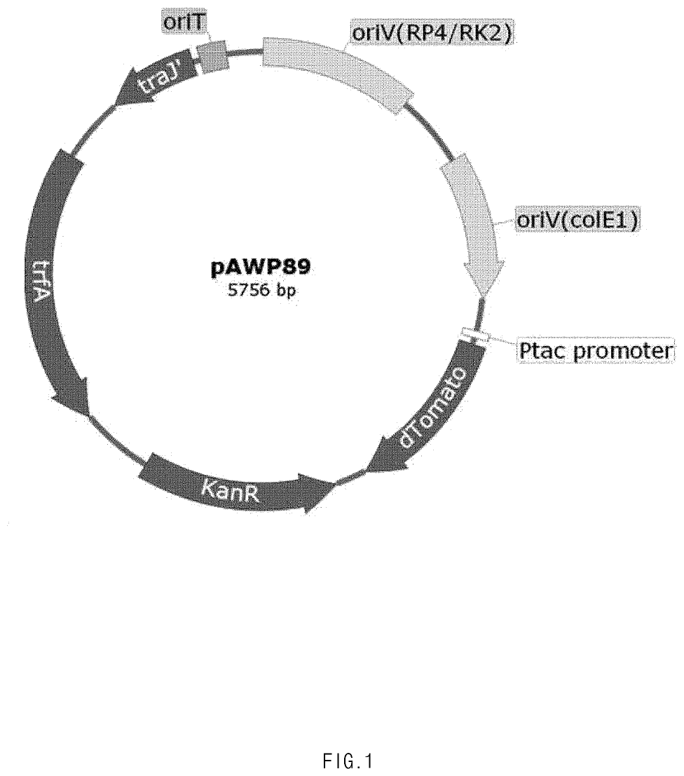

A pAWP89 vector was used. Details of the pAWP89 vector are described in Genetic Tools for the Industrially Promising Methanotroph Methylomicrobium buryatense (Appl Environ Microbiol. 2015 March; 81(5):1775-1781). (FIG. 1)

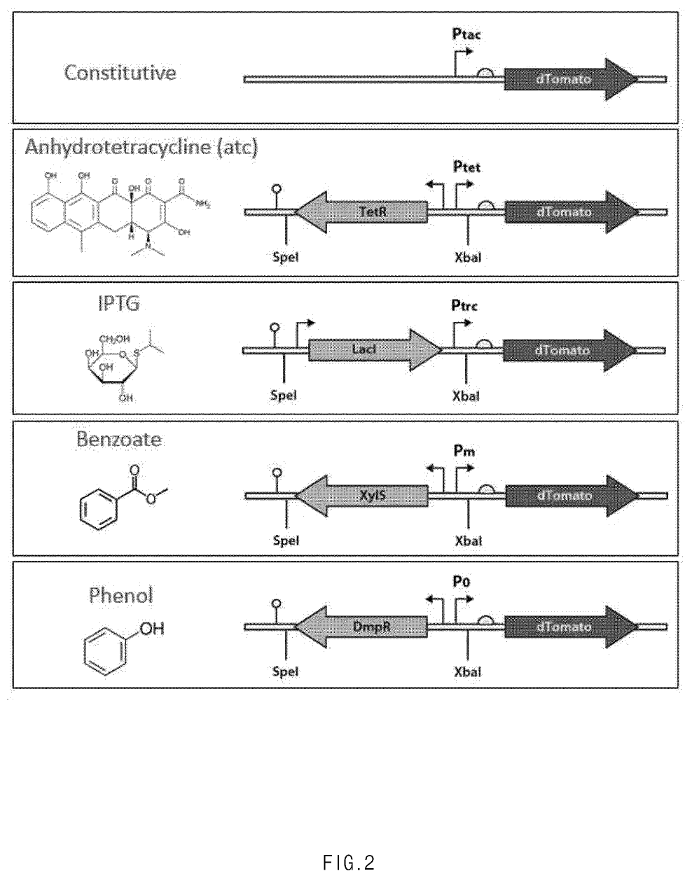

Plasmids were prepared by inserting TetR and Pₜₑₜ, LacI and P_{trc}, XylS and Pₘ, or DmpR and Pₒ into the pAWP89 vector by the following method. (FIG. 2)

1-1. Construction of pAWP89-XbaI

In order to facilitate cloning of genes other than dTomato present in a pAWP89 plasmid (Addgene #61264), an XbaI restriction enzyme site was inserted while the dTomato gene was removed. First, the pAWP89 plasmid was digested with NotI/HindIII to prepare a DNA fragment. Then, a DNA fragment amplified using the pAWP89 plasmid as a template and a primer pair, pAW89(NotI)-F1 and pAW89(NotI)-RI, containing the XbaI restriction enzyme site, and a DNA fragment amplified using the pAWP89 plasmid as a template and a primer pair, pAW89(HindIII)-F2 and pAW89(HindIII)-R2, containing the XbaI restriction enzyme site were prepared, respectively. These three DNA fragments were combined by the Gibson assembly method to construct a pAWP89-XbaI plasmid into which the XbaI restriction enzyme site was inserted.

1-2. Construction of pAWP89S-XbaI Plasmid

In order to facilitate promoter replacement while removing ColE1 origin of the pAWP89-XbaI plasmid, a SpeI restriction enzyme site was additionally inserted. Deleting the ColE1 origin reduces the size of the plasmid, increasing the efficiency of conjugation and preventing the growth inhibition of E. coli, a donor cell. First, the pAWP89-XbaI plasmid was digested with NotI/XbaI to prepare a DNA fragment. Then, a DNA fragment amplified using the pAWP89-XbaI plasmid as a template and a primer pair, pAWP89(NotI)-F1 and pAWPS(NotI)-R1, containing a SpeI restriction enzyme site, and a DNA fragment amplified using the pAWP89-XbaI plasmid as a template and a primer pair, pAWPS(XbaI)-F2 and pAWPS(XbaI)-R2, containing the SpeI restriction enzyme site, were prepared respectively. These three DNA fragments were combined by the Gibson assembly method to construct a pAWP89S-XbaI plasmid.

1-3. Construction of pAWP89S-Pₜₑₜ-dTomato Plasmid

To construct a plasmid expressing a dTomato protein by a Pₜₑₜ promoter, the pAWP89S-XbaI plasmid was digested with SpeI/XbaI to prepare a DNA fragment. Then, by performing amplification using pSEVA221-Pₜₑₜ-dCas9 plasmid (see SEQ ID NO: 40) as a template and a primer pair, pTet-dTomato(SpeI)-F1 and pTet-dTomato(SpeI)-R1, a DNA fragment containing TetR and Ptet was prepared. Also, by performing amplification using the pAWP89 plasmid as a template and a primer pair, pTet-dTomato(XbaI)-F2 and pTet-dTomato(XbaI)-R2, a DNA fragment was prepared. These three DNA fragments were combined by the Gibson assembly method to construct a pAWP89S-Pₜₑₜ-dTomato plasmid (see SEQ ID NO: 41).

1-4. Construction of pAWP89S-Ptrc-dTomato Plasmid

In order to replace the Pₜₑₜ promoter of the pAWP89S-Pₜₑₜ-dTomato plasmid with the P_{trc} promoter, the pAWP89S-Pₜₑₜ-dTomato plasmid was digested with SpeI/XbaI to prepare a DNA fragment. Then, a DNA fragment containing LacI and Ptrc was prepared by performing amplification using a pSEVA234 plasmid (Silva-Rocha, Martinez-Garcia et al. 2013) as a template and a primer pair, pAWPS-Ptrc(SpeI)-F and pAWPS-Ptrc(XbaI)-R. The pSEVA234 plasmid may be sequenced from NCBI's GeneBank accession number (KC847292). These two DNA fragments were combined by the Gibson assembly method to construct a pAWP89S-Ptrc-dTomato plasmid (see SEQ ID NO: 42).

1-5. Construction of pAWP89S-Pm-dTomato Plasmid

In order to replace the Pₜₑₜ promoter of the pAWP89S-Pₜₑₜ-dTomato plasmid with the Pₘ promoter, the pAWP89S-Pₜₑₜ-dTomato plasmid was digested with SpeI/XbaI to prepare a DNA fragment. Then, a DNA fragment containing XylS and Pₘ was prepared by performing amplification using the pSEVA228 plasmid (Silva-Rocha, Martinez-Garcia et al. 2013) as a template and a primer pair, pAWPS-Pm(SpeI)-F and pAWPS-Pm(XbaI)-R. The pSEVA228 plasmid may be sequenced from NCBI's GeneBank accession number (JX560388). These two DNA fragments were combined by the Gibson assembly method to construct a pAWP89S-Pm-dTomato plasmid (see SEQ ID NO: 43).

1-6. Construction of pAWP89S-Pₒ-dTomato Plasmid

In order to replace the Pₜₑₜ promoter of the pAWP89S-Pₜₑₜ-dTomato plasmid with the Pₒ promoter, the pAWP89S-Pₜₑₜ-dTomato plasmid was digested with SpeI/XbaI to prepare a DNA fragment. Then, a DNA fragment containing DmpR and Pₒ (see SEQ ID NO: 45) was prepared by performing amplification using a pSEVA131-phGESS4-sf plasmid (see SEQ ID NO: 44) as a template and a primer pair, pAWPS-PO(SpeI)-F and pAWPS-PO(XbaI)-R. These two DNA fragments were combined by the Gibson assembly method to construct a pAWP89S-Pₒ-dTomato plasmid (see SEQ ID NO: 46).

1-7. Construction of pAWP89S-Po-dTomato-RBS(Mdh), pAWP89S-Po-dTomato-RBS(mmoX), pAWP89S-Po-dTomato-RBS(mmoY), pAWP89S-Po-dTomato-RBS(mmoZ), pAWP89S-Po-dTomato-RBS(pmoA1), pAWP89S-Po-dTomato-RBS(pmoB1), pAWP89S-Po-dTomato-RBS(pmoC1), pAWP89S-Po-dTomato-RBS(pmoC2), pAWP89S-Po-dTomato-RBS(pmoC3) Plasmids

In order to replace the RBS regulating the translation of the dTomato protein of the pAWP89S-Pₒ-dTomato plasmid with RBS(mdh), the pAWP89S-Pₒ-dTomato plasmid was digested with PstI to prepare a DNA fragment. Then, a DNA fragment was prepared by performing amplification using the pAWP89S-Po-dTomato plasmid as a template and a primer fair, DmpR(PstI)-F and RBS-R, and a DNA fragment containing RBS(mdh) was prepared by performing amplification using the dTomato plasmid as a template and a primer fair, RBS(MDH)-F [symbol] dTomato(PstI)-R. These three DNA fragments were combined by the Gibson assembly method to construct a pAWP89S-Pₒ-dTomato-RBS (mdh) plasmid (see SEQ ID NO: 49).

The pAWP89S-Pₒ-dTomato-RBS(mmoX) plasmid (see SEQ ID NO: 50) was constructed by using a DNA fragment that was amplified from a DNA sequence containing RBS(mmoX) using the pAWP89S-Pₒ-dTomato plasmid as a template and a primer fair, RBS(MmoX)-F and dTomato(PstI)-R, in the same way as above.

The pAWP89S-Pₒ-dTomato-RBS(mmoY) plasmid (see SEQ ID NO: 51) was constructed using a DNA fragment that was amplified from a DNA sequence containing RBS(mmoY) using the pAWP89S-Pₒ-dTomato plasmid as a template and a primer pair, RBS(MmoY)-F and dTomato(PstI)-R, in the same way as above.

The pAWP89S-Pₒ-dTomato-RBS(mmoZ) plasmid (see SEQ ID NO: 52) was constructed using a DNA fragment that was amplified from a DNA sequence containing RBS(mmoZ) using the pAWP89S-Pₒ-dTomato plasmid as a template and a primer pair, RBS(mmoZ)-F and dTomato(PstI)-R, in the same way as above.

The pAWP89S-Pₒ-dTomato-RBS(pmoA1) plasmid (see SEQ ID NO: 53) was constructed using a DNA fragment that was amplified from a DNA sequence containing RBS(pmoA1) using the pAWP89S-Pₒ-dTomato plasmid as a template and a primer pair, RBS(PmoA1)-F and dTomato(PstI)-R, in the same way as above.

The pAWP89S-Pₒ-dTomato-RBS(PmoB1) plasmid (see SEQ ID NO: 54) was constructed using a DNA fragment that was amplified from a DNA sequence containing RBS(PmoB1) using the pAWP89S-Pₒ-dTomato plasmid as a template and a primer pair, RBS(PmoB1)-F and dTomato(PstI)-R, in the same way as above.

The pAWP89S-Pₒ-dTomato-RBS(pmoC1) plasmid (see SEQ ID NO: 55) was constructed using a DNA fragment that was amplified from a DNA sequence containing RBS(pmoC1) using the pAWP89S-Pₒ-dTomato plasmid as a template and a primer pair, RBS(pmoC1)-F and dTomato(PstI)-R, in the same way as above.

The pAWP89S-Pₒ-dTomato-RBS(pmoC2) plasmid (see SEQ ID NO: 56) was constructed using a DNA fragment that was amplified from a DNA sequence containing RBS(pmoC2) using the pAWP89S-Pₒ-dTomato plasmid as a template and a primer pair, RBS(pmoC2)-F and dTomato(PstI)-R, in the same way as above.

The pAWP89S-Pₒ-dTomato-RBS(pmoC3) plasmid (see SEQ ID NO: 57) was constructed using a DNA fragment that was amplified from a DNA sequence containing RBS(pmoC3) using the pAWP89S-Pₒ-dTomato plasmid as a template and a primer pair, RBS(pmoC3)-F and dTomato(PstI)-R, in the same way as above.

1-8. Construction of pAWPS-Pₒ-PtIspS-MVA Plasmid

To construct pAWPS-Pₒ-PtIspS-MVA, mevalonate pathway gene and PAWPS vector backbone were first prepared by PCR amplification using the corresponding primer pairs from pTSN-CcBisa-Mm and pAWPS-Pₒ-dTomato. The pTSN-CcBisa-Mm plasmid is pTrc99A, which contains mvaE and mvaS from Enterococcus faecalis, mvaK1 from Methanosarcina mazei, mvaK2 and mvaD from Streptococcus pneumoniae, E. coli robust inducible expression system, ispA, the codon-optimized CcBOS from Cynara cardunculus var. Scolymus. After assembling these two fragments, the resulting construct was used as a vector backbone, followed by PCR amplification. Isoprene synthase from Populus trichocarpa was amplified from pT-IspS, the pTrc99A plasmid containing codon-optimized PtIspS, and two fragments were assembled to generate pAWPS-Pₒ-PtIspS-MVA.

The primer sequences used in Experimental Example 1 are disclosed in Table 1 below.

TABLE 1

| Seq | | |
|---|---|---|
| ID | name | sequence |
| 23 | pAW89(NotI)-F1 | agccgtgtgc gagacaccgc |
| 24 | pAW89(NotI)-R1 | cccgacaatc tagatagctg tttcctgtgt gaa |
| 25 | pAW89(HindIII)-F2 | cagctatcta gattgtcggg aagatgcgtg at |
| 26 | pAW89(HindIII)-R2 | agtctggaaa gaaatgcata |
| 27 | pAWPS(NotI)-R1 | ttcagagact agtttcgcaa cctagtgaat gag |
| 28 | pAWPS(XbaI)-F2 | ttgcgaaact agtctctgaa atgagctgtt gac |
| 29 | pAWPS(XbaI)-R2 | tcacgcatct tcccgacaat |
| 30 | pTet-dTomato(SpeI)- | tcattcacta ggttgcgaaa tcagagattt tgagacacaa |
| | F1 | |
| 31 | pTet-dTomato(SpeI)- | tgctcaccat atgtatatct ccttcttaaa |
| | R1 | |
| 32 | pTet- | agatatacat atggtgagca agggcgagga |
| | dTomato(XbaI)-F2 | |
| 33 | pTet- | tcacgcatct tcccgacaat ctacttgtac agctcgtcca |
| | dTomato(XbaI)-R2 | |
| 34 | pAWPS-Ptrc(SpeI)-F | ctatcaacag gagtccaaga ctagtttgac accatcgaat |
| | | ggtgc |
| 35 | pAWPS-Ptrc(XbaI)- | aaagttaaac aaaattattt ctagagtgtg aaattgttat ccgct |
| | R | |
| 36 | pAWPS-Pm(SpeI)-F | ctatcaacag gagtccaaga ctagtacgtt cgtaatcaag |
| | | ccact |
| 37 | pAWPS-Pm(XbaI)-R | aaagttaaac aaaattattt ctagacatgg tcatgactcc attat |
| 38 | pAWPS-PO(SpeI)-F | ctatcaacag gagtccaaga ctagtagcgg ataacaattt |
| | | cacac |
| 39 | pAWPS-PO(XbaI)-R | aaagttaaac aaaattattt ctagatctcc aggttggcgg attgc |
| 62 | DmpR(PstI)-F | ggccattgct gaaacctgca |
| 63 | RBS-R | tctagatctc caggttggcg |
| 64 | dTomato(PstI)-R | gatcagcgtg ccgtcctgca |
| 65 | RBS(MDH)-F | cgccaacctg gagatctaga tgcagggtcg gcatcaatca |
| | | ttcttggagg agacacatgg tgagcaaggg cgagga |
| 66 | RBS(MmoX)-F | cgccaacctg gagatctaga catcattcat agaatgtgtt |
| | | acggaggaaa caagtaatgg tgagcaaggg cgagga |
| 67 | RBS(MmoY)-F | cgccaacctg gagatctaga cgttcgatca acctcaaacc |
| | | aaaaaggaac atcgatatgg tgagcaaggg cgagga |
| 68 | RBS(MmoZ)-F | cgccaacctg gagatctaga tggacacttt ttcaacggcc |
| | | tgttaaggag aatgacatgg tgagcaaggg cgagga |
| 69 | RBS(PmoA1)-F | cgccaacctg gagatctaga gaaaataaga aaacgacaaa |
| | | tttggaggta actttaatgg tgagcaaggg cgagga |
| 70 | RBS(PmoB1)-F | cgccaacctg gagatctaga gtgacccaac agcaagaact |
| | | cgaaagagga gagatcatgg tgagcaaggg cgagga |
| 71 | RBS(PmoC1)-F | cgccaacctg gagatctaga ggcccctgtc aaccatcact |
| | | ttaggaggaa caaacaatgg tgagcaaggg cgagga |
| 72 | RBS(PmoC2)-F | cgccaacctg gagatctaga ccggtgttca catcgatata |
| | | acagttggag gtaaaaatgg tgagcaaggg cgagga |
| 73 | RBS(PmoC3)-F | cgccaacctg gagatctaga gtggcatcaa tccaacacca |
| | | actcaggaga aatcagatgg tgagcaaggg cgagga |
| 74 | sgRNA_F | GGCACGATCCCTGTAACTAG |
| 75 | sgRNA_R | AATGAAGCCGTCGGAAAAC |
| 76 | mmoX_R | GCTTCATGCCCTTCCACAG |
| 77 | sgRNA(AgeI)-F | agatattattgaaaaggagaccgg |
| 78 | sgRNA(PacI)-R | ttttatttgatgcctttaattaatcgaggc |
| 79 | pAWPS-Po_VF | ttctacaaactcttattgtcgggaagatgcgt |
| 80 | pAWPS- | ggatacagagcaagccatatgtatatctccttcttaaagttaaa |
| | Po(PtIspS)_VR | |
| 81 | PtIspS-MVA- | agatatacatatggcttgctctgt |
| | DAAT_IF | |
| 82 | IspS_CcBOS_I1R | ttcctgtgtgaattgcgttgcgctctaga |
| 83 | MVA_I2F | caacgcaattcacacaggaaacagc |
| 84 | MVA_DAAT_IR | atcttcccgacaataagagtttgtagaaac |

### Experimental Example 2: Conjugation by Triparental Mating

2-1. Cultivation of Methanotrophs (3 Days Before Mating)

M. capsulatus Bath cell stock was inoculated into 3 ml of NMS media, covered with a rubber stopper, and sealed with an aluminum seal. 50% methane gas was supplied in the headspace of the vial, and seed culture was performed at 37° C. and 200 rpm for 24 hours. M. trichosporium OB3b strain was seed cultured in the same manner as in the Bath, except that the culture condition was 30′° C.

2-2. Cultivation of Methanotrophs (2 Days Before Mating)

The M. capsulatus bath was inoculated with 5% seed in NMS media containing 10 μM CuCl₂, covered with a rubber stopper, and sealed with an aluminum seal. 50% methane gas was supplied in the headspace and incubated at 37° C. and 200 rpm for 19 hours. M. trichosporium OB3b was subjected to main culture under the same conditions except that the culture condition was 30° C. for 30 hours.

2-3. Preparation of Donor Strain and Helper Strain (1 Day Before Mating)

E. coli CC118 containing the pAWP89S-Pₒ-dTomato, pAWP89S-Pₘ-dTomato, or pAWP89S-P_{trc}-dTomato plasmid was inoculated in 10 ml of LB media containing Kan 5 μg/ml, respectively, and cultured at 37° C. and 200 rpm for 19.5 hours. E. coli HB101, a helper strain for conjugation, was cultured in 10 ml of LB media at 37° C., 200 rpm, and 19.5 hours.

2-4. Mating

The OD₆₀₀ value of each strain was measured and mixed in a 50 ml tube so that the OD₆₀₀ value based on 1 ml was 3. The order of introduction was as follows: donor strain, helper strain, and methanotrophs (recipient).

After centrifugation of the strain mixture at the condition of 1000 g for 20 min, the supernatant was removed, and the strain was resuspended in 200 μl of NMS and spread on a NMS+0.12% nutrient agar plate.

2-5. Strains Screening by Single Colony Isolation

The spread strains were mated at 37° C. or 30° C. for 3 days, and the cells were harvested with 500 μl of NMS, and diluted 10, 100, 1,000 and 10,000 times, and then, 100 μl of the cells were spread on a NMS-Kan (25 μg/ml) plate, and the transconjugated methanotrophs were selected. In the case of the M. capsulatus Bath, a single colony isolation was performed using a NMS-Kan (25 μg/ml) plate, and cells were cultured at 37° C. for 6 days, and in the case of M. trichosporium OB3b, a selection process was performed by culturing the strain at 30° C. for 10 to 11 days. In the case of M. capsulatus Bath, transconjugates that were not contaminated with the donor or the helper were obtained through this selection process, and in the case of M. trichosporium OB3b, the selection process was repeated two more times to secure transconjugates through a total of three selection processes.

### Experimental Example 3: Cultivation, Fluorescence Analysis, and SDS-PAGE Analysis of Transconjugated Methanotrophs

3-1. Cultivation of Transformed Methanotrophs

The transconjugated M. capsulatus Bath and M. trichosporium OB3b were inoculated by 10% in NMS medium containing 25 μg/ml kanamycin and 10 μM CuCl₂. For the methanotrophs transconjugated with the pAWP89S-Pₒ-dTomato, 10 μM of phenol was contained in the medium. After sealing, 50% methane gas was substituted in the headspace. The recombinant M. capsulatus Bath was cultured at 37° C. for 24 hours or 44 to 48 hours, and therecombinant M. trichosporium OB3b was cultured at 30° C.

3-2. Fluorescence Analysis

For analysis of the expression level by fluorescence, 200 μl was sampled in a 96-well plate and a Tecan microplate reader (excitation: 535 nm, emission: 590 nm) was used. FACS analysis was performed using BD Biosciences FACSCalibur equipment (excitation: 488 nm, emission: 585 nm).

3-3. SDS-PAGE Analysis

The cultured cells were lysed by ultrasonic waves and samples were prepared for SDS-PAGE. The protein concentration of the sample was 0.36 mg/ml in a control group not treated with phenol, and 0.30 mg/ml in the experimental group treated with 10 μM phenol.

5% stacking gel and 15% running gel were prepared and mounted on an electrophoresis device. In the case of M. capsulatus Bath, a sample of 5 μg/μl was loaded and electrophoresis was performed at 100V for 90 minutes. For M. trichosporium OB3b, a sample of 10 μg/μl was loaded and electrophoresis was performed at 100V for 110 minutes.

### Experimental Example 4: Production, Toxicity Test, Analysis, and Quantification of Isoprene

4-1. Toxicity Test of Isoprene and Epoxy Isoprene

To analyze the toxicity of isoprene and epoxyisoprene using wild-type strains, 2 mL cultures were transferred to 150 mL serum bottle containing 19 ml NMS medium with or without 10 μM CuCl₂. After incubation for 6.5 to 7.5 hours, an appropriate amount of isoprene or epoxy isoprene (2-methyl-2-vinyloxirane) diluted in dodecane was added. The bottle was sealed, 50% of the headspace was replaced with methane, and the bottle was further incubated. Sampling of 1 mL cultures for cell density measurement was performed at appropriate time intervals using a disposable syringe without reopening the bottle.

4-2. Fermentation

A benchtop stirred tank reactor (STR, CNS21-M05-001, BIOCNS, Korea) equipped with a ceramic microsparger and low-temperature dodecane trap was operated for isoprene production. The working volume and total volume of the reactor were 0.5 L and 1 L, respectively. A modified NMS medium containing 3 times nitrate, 1.5 times phosphate, and 3 times trace metals (300 μL of 10,000× trace metal stock solution (5 g/L FeSO4·7H2O, 4 g/L ZnSO4·7H2O, 0.2 g/L MnCl2·4H2O, 0.5 g/L CoCl2·6H2O, 0.1 g/L NiCl2·6H2O, 0.15 g/L H3BO3, 6.84 mL of 0.5 M EDTA)) in a basic methanotroph culture medium was used for fermentation. All equipment were autoclaved at 121° C. for 15 minutes. 5 to 6 hours after inoculation, cells were induced to express the isoprene synthase group with 5 μM phenol. The flow rate of gas mixture (methane:air=50:50) was maintained at 0.04 vvm for the first 15 hours. Then, the gas composition was changed to methane:air=20:80 and the flow rate was gradually increased to 0.25 vvm. During the operation of the reactor, temperature and pH were maintained at 37° C. and 6.9, respectively. For fermentation with high cell density, nitrate concentration was measured by kit based spectrometric assay (NO3(N)-CA, HUMAS, Korea), and then nitrate was periodically supplied in the form of 3.76 M KNO₃ solution.

4-3. Analysis and Quantification of Isoprene

Isoprene was analyzed with a GC-mass spectrometer (GC-MS; 5977A MSD, Agilent Technologies) equipped with an HP-5MS column (30 m×0.25 mm×0.25 μm; Agilent Technologies). 5 μL of sample was injected for detection of isoprene dissolved in dodecane. Column flow rate was 1 mL/min. The injector temperature was maintained at 260° C., the oven temperature was maintained at 50° C. for 2 minutes, and the isoprene sample dissolved in dodecane was post-treated at 250° C. for 30 minutes. A scanning mode in the range of 30 to 100 m/z and selective ion monitoring at 67 m/z were used for isoprene detection. The samples collected from the cold dodecane trap were used for analysis for isoprene quantification in the bioreactor. The isoprene produced during a bioreactor operation was calculated according to the following Equations (1) to (5):

K_{gas/oil}·C_{isp,oil}=C_{isp,g} (1)
where K_{gas/oil} is the experimentally determined equilibrium constant of isoprene between gas and oil phases in the dodecane trap. C_{isp, oil} is the concentration (mol/L) of isoprene in the oil phase determined from GC-MS analysis, and C_{isp,g} is the concentration (mol/L) of isoprene in the gas phase;

C_{isp,g}·Q_{g}=Pᵢₛₚ (2)
where Q_{g} is the off-gas flow rate (L/min) measured during reactor operation and Pᵢₛₚ is the isoprene productivity (mol/min);

$${P}_{isp} \cdot \frac{1}{{V}_{L}} = {P}_{isp , vol} \qquad ( 3 )$$

where V_{L} is the working volume (L) and P_{isp,vol} is the isoprene production per unit volume (mol/(L·min));

ΔP_{isp,vol}·Δt=ΔC_{isp,broth} (4)
where ΔP_{isp,vol} is the production of isoprene per average volume between mol/(L·min) at a sampling point, Δt is the time difference between sampling points (minutes), and ΔC_{isp,broth} is an amount (mol/L) of isoprene produced in the reactor between those sampling time points. The total amount of isoprene C_{isp,broth} produced during fermentation may then be calculated as summation.

ΣΔC_{isp,broth}=C_{isp,broth} (5)

4-4. Analysis of Organic Acids

Organic acids were analyzed on an HPLC system (Agilent Technologies) equipped with an Aminex HPX-87H column (300 mm×7.8 mm; BioRad). Acetate, succinate, malate, and formate were detected using a refractive index detector, and mevalonate was analyzed using a wavelength detector at 210 nm. Samples were prepared from supernatants of cell culture aliquots filtered through 0.2-μm membranes.

### Example 1: Confirmation of dTomato Expression in M. trichosporium OB3b Transconjugated with a pAWP89S-Pₜₑₜ-dTomato Plasmid

M. trichosporium OB3b transconjugated with the pAWP89S-Pₜₑₜ-dTomato plasmid was treated with anhydrotetracycline (aTc) at 0 μM, 0.5 μM, 1 μM, or 2 μM, respectively, to induce expression, and the expression of the fluorescent protein dTomato was examined for 8 days. The experiment was performed by the method of Experimental Example 3 above.

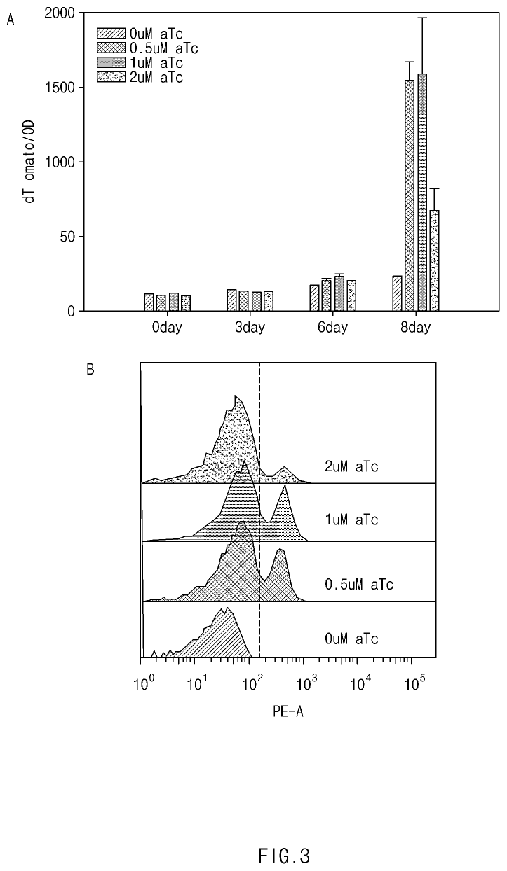

According to (A) in FIG. 3, fluorescence intensity was the highest in the groups treated with 0.5 μM and 1 μM of aTc, and rather decreased in the group treated with 2 μM. Therefore, it was confirmed that the optimal treatment concentration of aTc was 0.5 to 1 μM. In addition, the time required for expression to be most active was about 8 days.

According to (B) in FIG. 3 showing FACS analysis results, two peaks were formed based on an one-dotted chain line, which means that there are a significant number of strains expressing dTomato as well as strains that do not express dTomato. Although not clearly identified, it is considered that this is because aTc is difficult to reach the vector inside M. trichosporium OB3b and thus cannot uniformly induce expression of the Pₜₑₜ promoter.

### Example 2: Confirmation of dTomato Expression in M. capsulatus Bath Transconjugated with a pAWP89S-P_{trc}-dTomato, pAWP89S-Pₒ-dTomato, or pAWP89S-Pₘ-dTomato Plasmid

2-1. Confirmation of dTomoto Expression by Fluorescence Measurement

The fluorescence expression of dTomato was measured in the same manner as in Example 1 above.

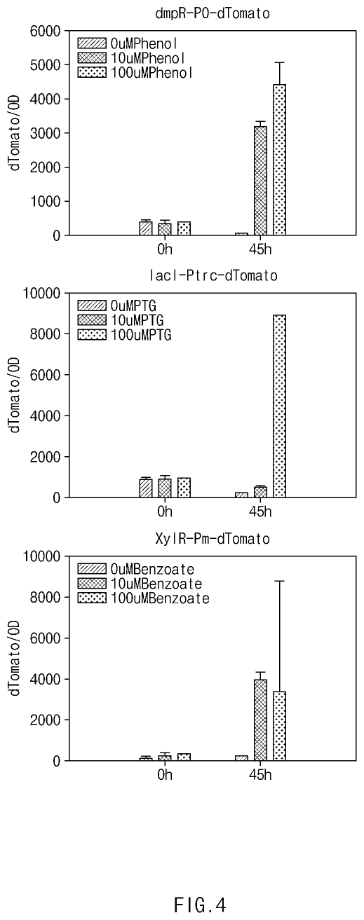

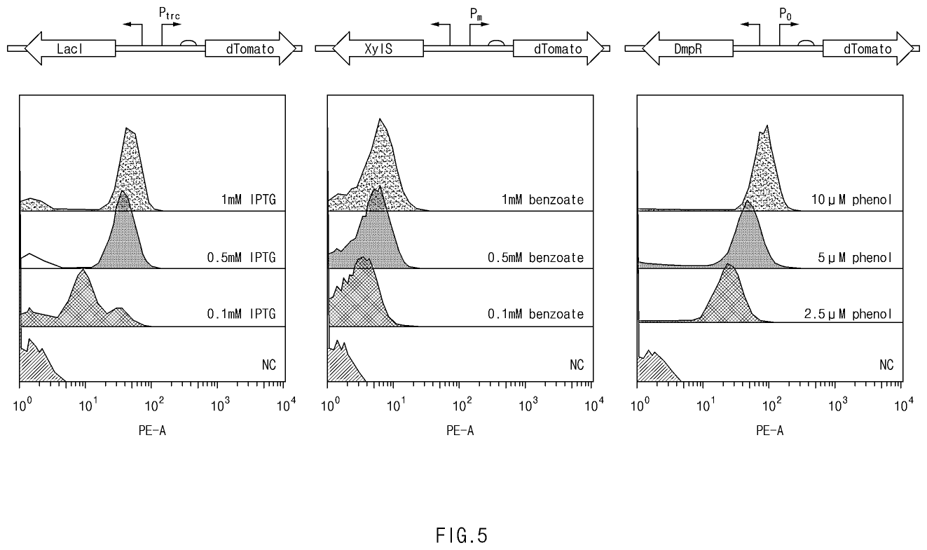

According to FIGS. 4 and 5, the M. capsulatus Bath harboring pAWP89S-Pₒ-dTomato plasmid showed the highest fluorescence value (dTomato/OD₆₀₀ₙₘ) after 45 hours compared to a group in which the pAWP89S-P_{trc}-dTomato plasmid and a group in which the pAWP89S-Pₘ-dTomato plasmid was introduced.

Specifically, although the strain harboring either pAWP89S-P_{trc}-dTomato or pAWP89S-Pₘ-dTomato plasmid was treated with a higher concentration of inducer (0.1 to 1 mM IPTG or 0.1 to 1 mM benzoate) than the strain with the pAWP89S-Pₒ-dTomato plasmid, the strains showed lower fluorescence values.

However, in the strain in which the pAWP89S-Pₒ-dTomato plasmid was introduced, it was observed that the strain growth in the group to which 100 μM phenol was treated was inhibited more than that in the strain more than the group to which 10 μM phenol was treated.

2-2. Confirmation of dTomato Expression by SDS-PAGE

The expression level of dTomato was measured by the SDS-PAGE method of Example 3.

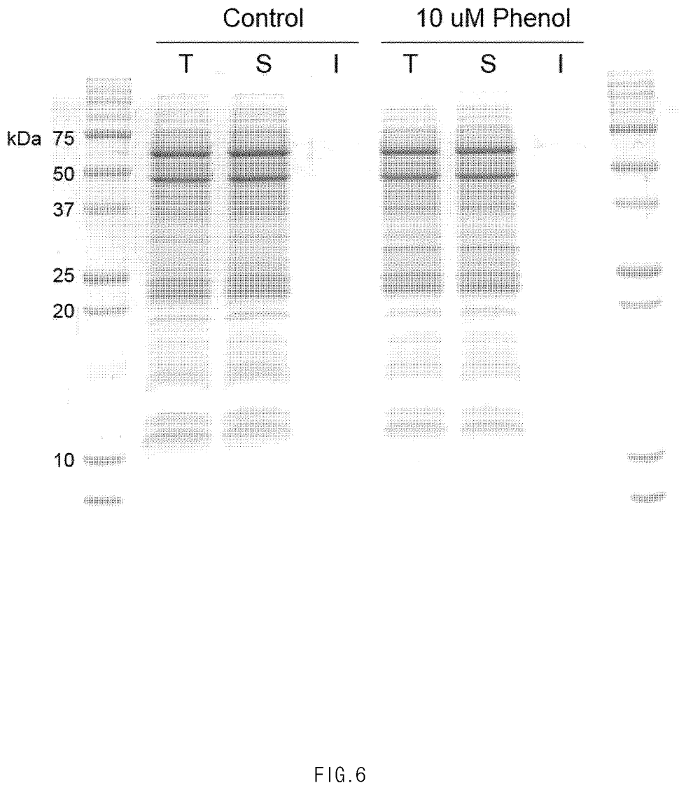

According to FIG. 6, a band of 26.97 kDa corresponding to dTomato, which was not seen in the control group, was clearly shown in in the experimental group treated with 10 μM of phenol. As a result of the experiment, it was confirmed that the vector containing the DmpR and Po promoters induced expression of the target protein in the M. capsulatus Bath, which was observed by bands on the SDS-PAGE.

2-3. Confirmation of Inhibition of Leaky Expression by Tight Regulation of M. capsulatus Bath in which a pAWP89S-Pₒ-dTomato Plasmid was Introduced

M. capsulatus Bath harboring pAWP89S-Pₒ-dTomato plasmid was cultured in a medium without phenol and a medium containing 10 μM phenol for 45 hours, respectively, and their dTomato fluorescence expressions were compared. According to FIG. 7, assuming that the dTomato expression level in the presence of 10 μM phenol was 100%, the relative dTomato expression level in the absence of phenol was only less than 2%, so it was confirmed that the inhibition of leakage expression was excellent.

2-4. Expression Time Under Conditions Induced by 10 μM Phenol

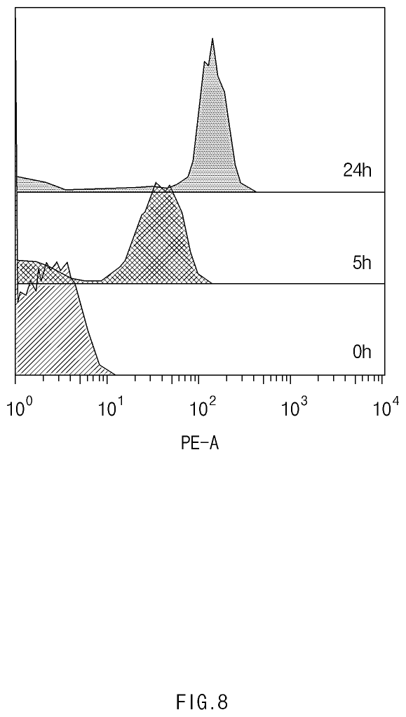

According to FIG. 8, in the M. capsulatus Bath harboring pAWP89S-Pₒ-dTomato plasmid, fluorescence due to dTomato expression was observed only 5 hours after the supply of 10 μM phenol.

### Example 3: Confirmation of dTomato Expression in M. trichosporium OB3b Tranconjugated with pAWP89S-P_{trc}-dTomato, pAWP89S-Pₒ-dTomato, or pAWP89S-Pₘ-dTomato Plasmids

3-1. Expression Confirmation by Fluorescence Measurement

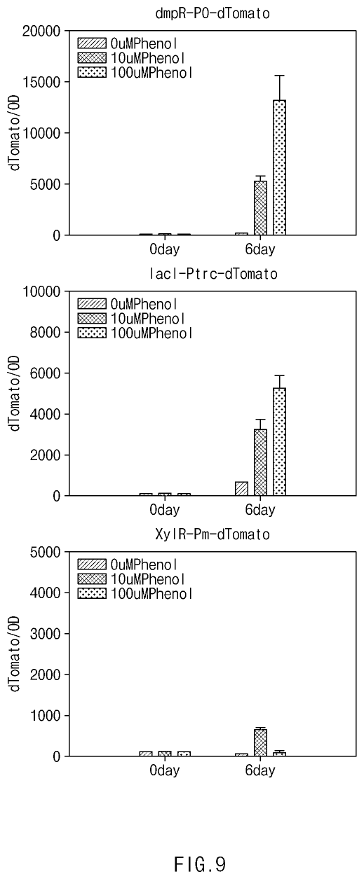

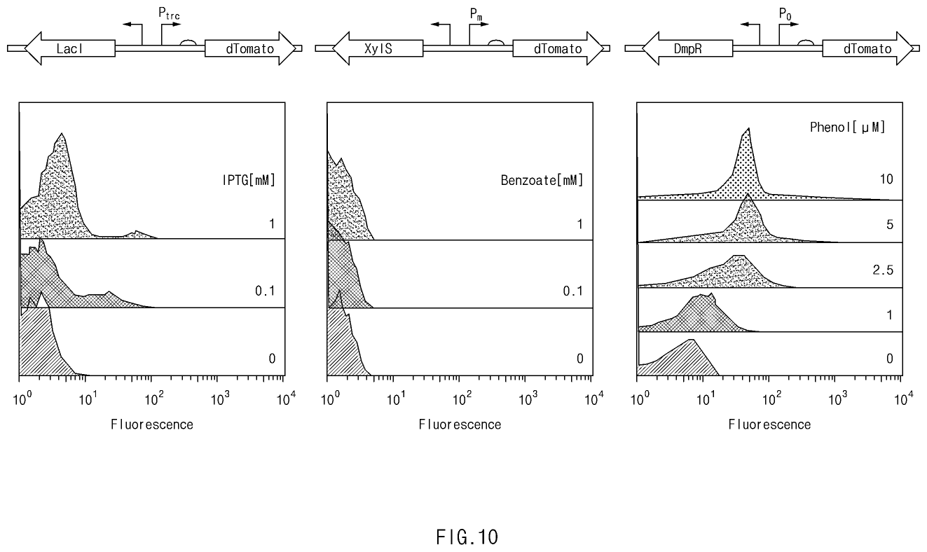

According to FIGS. 9 and 10, M. trichosporium OB3b harboring the pAWP89S-Pₒ-dTomato plasmid showed the highest fluorescence value (dTomato/OD₆₀₀ₙₘ) after 6 days than the group introduced with the pAWP89S-P_{trc}-dTomato plasmid and with the pAWP89S-Pₘ-dTomato plasmid.

Specifically, the strain in which the pAWP89S-P_{trc}-dTomato plasmid was introduced and the strain in which the pAWP89S-Pₘ-dTomato plasmid was introduced showed the lower fluorescence value (dTomato/OD₆₀₀ₙₘ) than that of the strain in which the pAWP89S-Pₒ-dTomato plasmid was introduced, even though the strains were treated with a larger amount of inducer (0.1 to 1 mM IPTG or 0.1 to 1 mM benzoate).

3-2. Confirmation of dTomato Expression by SDS-PAGE

The M. trichosporium OB3b harboring the pAWP89S-Pₒ-dTomato vector was introduced was subjected to the SDS-PAGE analysis under the same conditions as in 3-2 above.

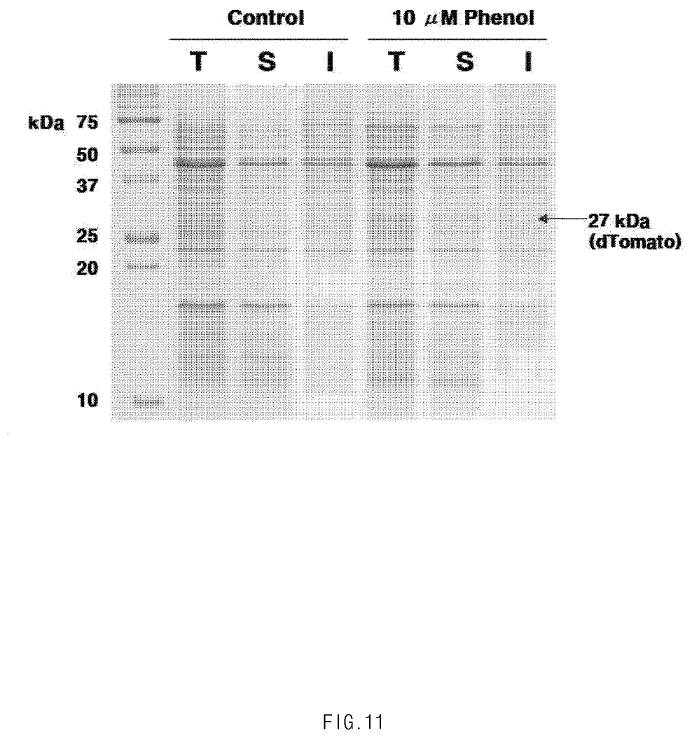

According to FIG. 11, the experimental group treated with 10 μM of phenol showed an additional band of 27 kDa corresponding to dTomato than the control group. As a result of the experiment, it was confirmed that the vector containing the DmpR and Pₒ promoters induced expression of the target protein in methanotrophs, which was observed as bands on the SDS-PAGE.

### Example 4: Confirmation of Activation of a DmpR Promoter by Benzene

It was confirmed whether dTomato could be induced with benzene under sMMO expression conditions (copper-deficient medium).

The M. capsulatus Bath with the pAWP89S-Pₒ-dTomato plasmid were cultured under sMMO expression condition (copper-deficient medium) and pMMO expression condition (copper-containing medium) while supplying 0.5 mM benzene for 24 hours. A strain supplied with 10 μM of phenol was used as a control group.

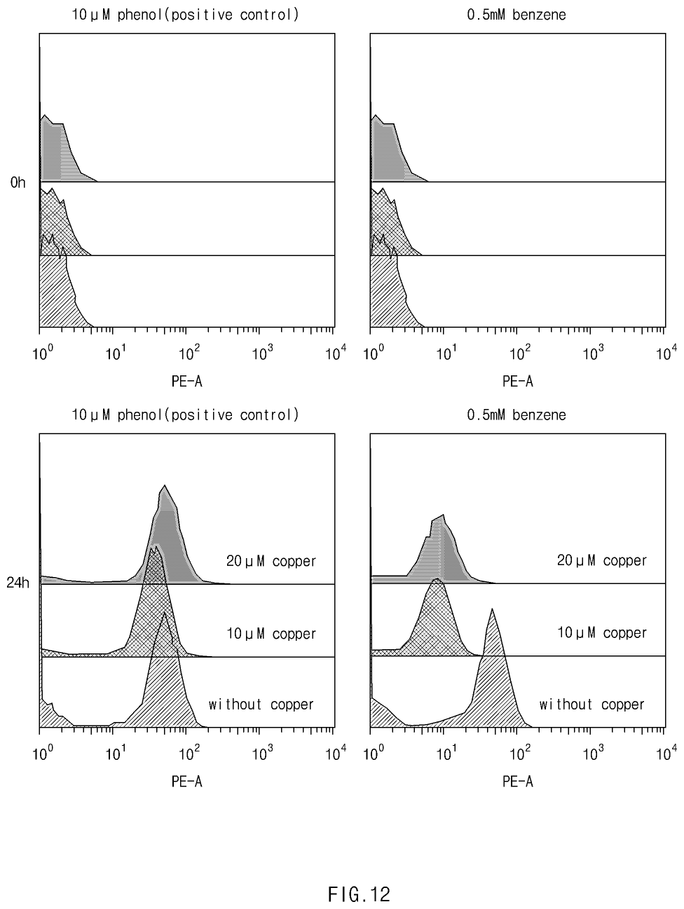

According to FIG. 12, the transconjugated strain cultured under the sMMO expression condition converted benzene to phenol by the sMMO enzyme activity, and dTomoto expression was highly induced as in the control group to which phenol was directly added. In the pMMO expression condition with copper, a relatively small fluorescence value was confirmed. This is an example showing that the developed expression system can be used as a sensor system that can identify the difference in sMMO and pMMO activities.

### Example 5: Regulation of Protein Expression by Using DmpR (Δ2-204) and Various RBS

To improve the expression system for methanotrophs of the disclosure, a constitutive expression system was prepared by removing A domain of DmpR. In addition, in order to regulate the expression level of the protein in various ways, various ribosomal binding sites derived from methanotrophs were combined with the Pₒ promoter to identify the expression level of the dTomato protein.

Specifically, an expression plasmid in which an existing T7 gene 10 ribosomal binding sites were replaced with the ribosomal binding sites derived from M. capsulatus Bath genes, mdh, mmoX mmoY, mmoZ, pmoA1, pmoB1, pmoC1, pmoC2 or pmoC3 were constructed. Thereafter, the M. capsulatus Bath transconjugated with each plasmid was cultured for 45 hours under the condition of supplying 0 μM or 10 μM phenol, and then the fluorescence of dTomato proteins was compared.

In the case of using various ribosomal binding sites derived from methanotrophs, the expression level of dTomato in using the ribosomal binding sites derived from mdh, pmoB1, and pmoC3 was higher than that in using the existing T7 gene 10, and in particular, the fluorescence value in using pmoB1 was 2.3 times higher than that in using the existing T7 gene 10. In addition, the dTomato fluorescence value in using mmoX mmoY, mmoZ, mmoC1, and mmoC2 ribosomal binding sites were various, but all of them were lower than that in using an existing ribosomal binding site. In addition, it was confirmed that the fluorescence values at the single cell level were uniformly distributed in all cases.

### Example 6: Confirmation of Industrial Usefulness (FmDTA Expression in M. capsulatus Bath)

A constitutive P_{tac} promoter-based (FmDTA-pAWP) or Pₒ promoter-based (Pₒ-FmDTA-pAWP) expression system was constructed to introduce highly valuable pharmaceutical raw material, D-threonine production enzyme (threonine aldolase derived from Filomicrobium marinum (FmDTA)) into methanotrophs.

As a result of culturing methanotrophs containing each plasmid and analyzing the expression level by SDS-PAGE, it can be confirmed that the expression level is remarkably high when the Pₒ system is used. As a reference, the band at the same position cannot be observed as expected in a negative control, methanotrophs containing a pAWP89 empty vector. This is an example showing that the newly constructed Pₒ system is useful for high expression of an enzyme of interest in methanotrophs, and is expected to be useful for the expression various enzymes.

### Example 7: Use of CRISPR/Cas9-Based Cytosine Base Editor in Methanotrophs

To verify the activity of a cytosine base editor using the Pₒ promoter in methanotrophs, a nCas9-BE(APOBEC-nCas9-UGI) plasmid and a pAWPSG-Pₒ-nCas9-BE-RiboJ-sgRNA(McglgA4) regulating sgRNA expression with the Pₒ promoter plasmid (see SEQ ID NO: 58) were constructed. In addition, to enhance sgRNA expression, a pAWPSG-Pₒ-nCas9-BE-pTac-RiboJ-sgRNA(McglgA4) plasmid (see SEQ ID NO: 59) was constructed by inserting a P_{tac} promoter in front of the sgRNA. 5′-GTTTCCAGCCGAGGCCGAGC-3′ was selected as the sgRNA sequence, and this sgRNA sequence binds to the glycogen synthase gene glgA1 to induce mutation of the TGG(Trp) codon to a stop codon. Thereafter, M. capsulatus Bath harboring the cytosine base editor plasmid was cultured in the presence of phenol, and the target sequence of glgA1 was analyzed by Sanger sequencing. In addition, in order to accumulate the mutation of the target sequence, three serial transfer culture was performed.

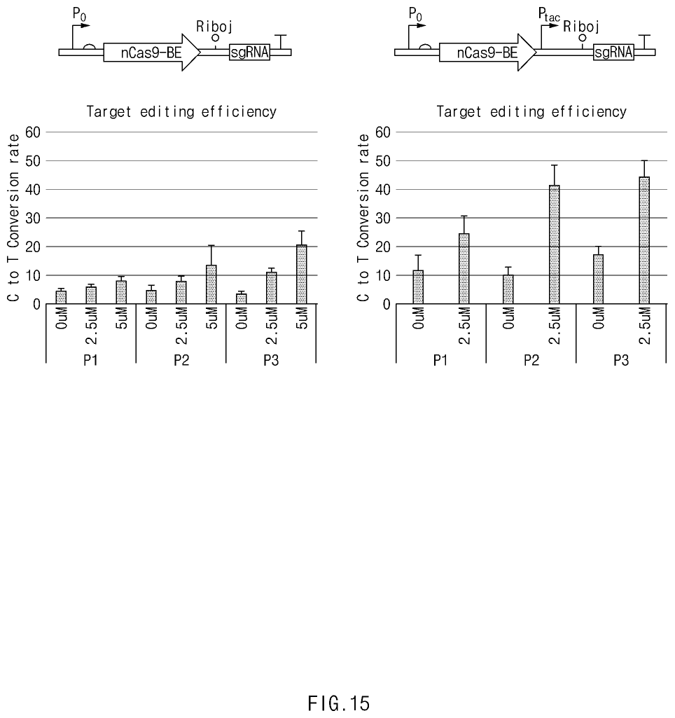

As a result, as shown in FIG. 15, it was confirmed that the target sequence was edited with thymine in more than 40% of the total cells when using the cytosine base editor system. In addition, it was found that the editing was performed more efficiently when the tac promoter was placed in front of the sgRNA, and it was confirmed that the point mutation was accumulated as subculture was performed. Through these results, it was confirmed that the cytosine base editor system using the Pₒ promoter operated well in methanotrophs, and the productivity of target substances could be expected to increase through the inhibition of glycogen biosynthesis.

### Example 8: Establishment of Isoprene Production Pathway

In order to induce the isoprene production in methanotrophs, the phenol-inducible Pₒ promoter-based gene expression system of the disclosure was applied.

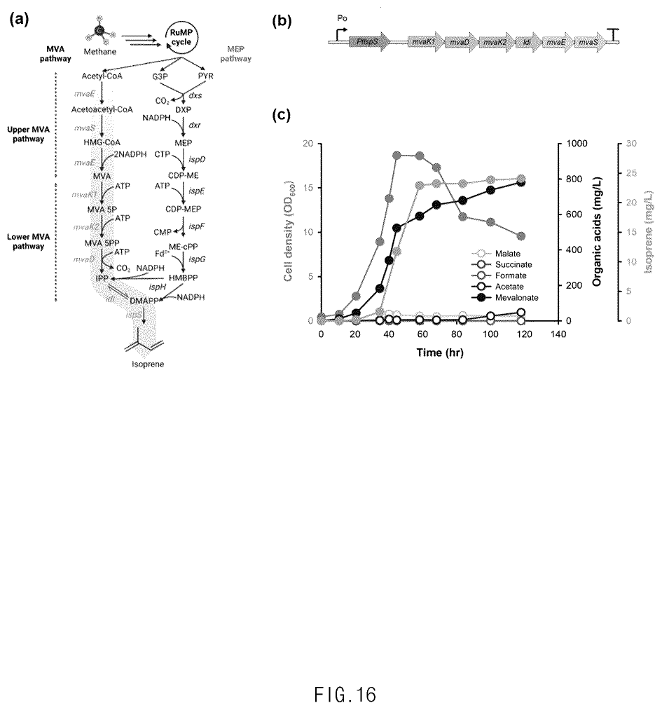

Specifically, as shown in (a) in FIG. 16, in order to produce isoprene through the mevalonate (MVA) pathway, a plasmid in which isoprene synthase (PtIsps) of P. trichocarpa and a MVA pathway gene (Pₒ-PtIspS-MVA) were inserted was introduced into the M. capsulatus Bath strain (Pₒ-PtIspS-MVA/Bath).

The Pₒ-PtIspS-MVA/Bath strain was fermented in modified NMS medium (3 times nitrate, 1.5 times phosphate buffer, and 3 times trace metal stock). As shown in (c) in FIG. 16, the Pₒ-PtIspS-MVA/Bath strain showed a maximum cell density (OD₆₀₀) of 18.7 and produced 24 mg/L of isoprene and 781 mg/L of mevalonate as an intermediate.

From the above results, when the expression of the MVA pathway was induced using the Pₒ promoter, it was confirmed that the methanotroph, M. capsulatus Bath strain produced significant amount of isoprene.

### Example 9: mmoX Knockout to Improve Isoprene Production

9-1. Confirmation of Toxicity of Isoprene and Epoxyisoprene

To examine the potential toxicity of isoprene produced in M. capsulatus Bath, growth assays were performed in the presence of various concentrations of isoprene while regulating the expression of sMMO and pMMO enzymes by controlling the addition of copper.

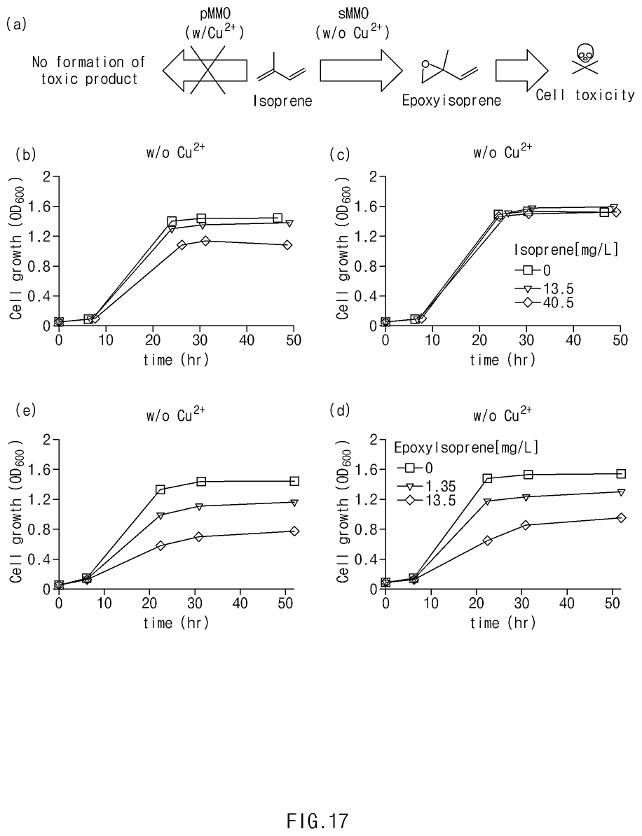

As a result, it was confirmed that the growth of the strain expressing the sMMO enzyme in the absence of copper was reduced with the isoprene addition ((b) in FIG. 17), whereas the growth of the cells expressing pMMO in the presence of copper was unaffected by isoprene ((c) in FIG. 17).

In addition, in order to examine whether the growth inhibition of the strain was due to the oxidation of isoprene by sMMO, the strain growth was analyzed in the presence of 1.35 and 13.5 mg/L of epoxyisoprene, an oxidation product of isoprene. As a result, it was confirmed that the growth of both the sMMO-expressing strain and the pMMO-expressing strain were reduced depending on the concentration of the epoxy isoprene treated above ((d) and (e) in FIG. 17). From these results, it can be seen that isoprene oxidation by sMMO is toxic to cells, resulting in inefficiency in converting methane to isoprene.

In the end, it was confirmed that the broad substrate specificity of sMMO in methanotrophs can be an obstacle to increase isoprene production, and this can be solved by expressing pMMO showing no isoprene activity by adding copper during the culture of methanotrophs.

9-2. Construction of mmoX Knockout Strains

During the high cell-density culture, copper can often be depleted, which can activate the expression of sMMO and interfere with isoprene production. Therefore, in order to improve isoprene production by preventing sMMO expression from being activated even in the above environment, mmoX knockout was constructed by introducing a premature stop codon into the mmoX gene encoding one of the sMMO subunits.

Specifically, mmoX among the endogenous genes of M. capsulatus Bath was targeted using a CRISPR/Cas9-based cytosine base editor in the methanotrophs of Example 7 described above. To construct a plasmid whose expression of nCas9-BE(APOBEC-nCas9-UGI) and sgRNA was regulated by the Pₒ promoter, first, pAWPSG-Pₜₑₜ-nCas9-BE-P_{tac}-sgRNA plasmid was constructed. A sgRNA cassette containing the P_{tac} promoter was synthesized, the synthesized gene was PCR-amplified with the corresponding primer pair (SEQ ID NOs: 74 and 75), and then digested by the AgeI/PacI restriction enzymes of the pAWPSG-Pₜₑₜ-nCas9-BE plasmid to be assembled with a constructed vector backbone. The pAWPSG-Pₜₑₜ-nCas9-BE plasmid was digested with SpeI/XbaI, and the Pₒ promoter was amplified from the pGESSv4 template using the corresponding primer pair to replace the Pₜₑₜ promoter with the Pₒ promoter. The vector backbone and the Po promoter were assembled to construct the pAWPSG-Pₒ-nCas9-BE-P_{tac}-sgRNA plasmid. Spacer sequences (mmoX_1 and mmoX_2) for the mmoX gene to be targeted are as follows.

TABLE 2

| SEQ | Target | | |
|---|---|---|---|
| ID | gene in | Sequence | Editing |
| NO. | spacer | (5′→3′) | description |
| 85 | mmoX_ | (+) | Gln (CAG)→ |
| | 1 | atgcaCaggaagtgcaccgt | Stop (TAG) |
| 86 | mmoX_ | (−) | Trp (TGG)→ |
| | 2 | atcCCacagcatcccggtag | Stop (TAA) |

Using sgRNA complexed with nCas9-CD(cytidine deaminase)-UGI(UNG inhibitor), a premature stop codon was induced by base editing of individual genes through subculture in the presence of 2.5 μM phenol. In order to accumulate the mutation in the target sequence, three serial transfer cultures were performed.

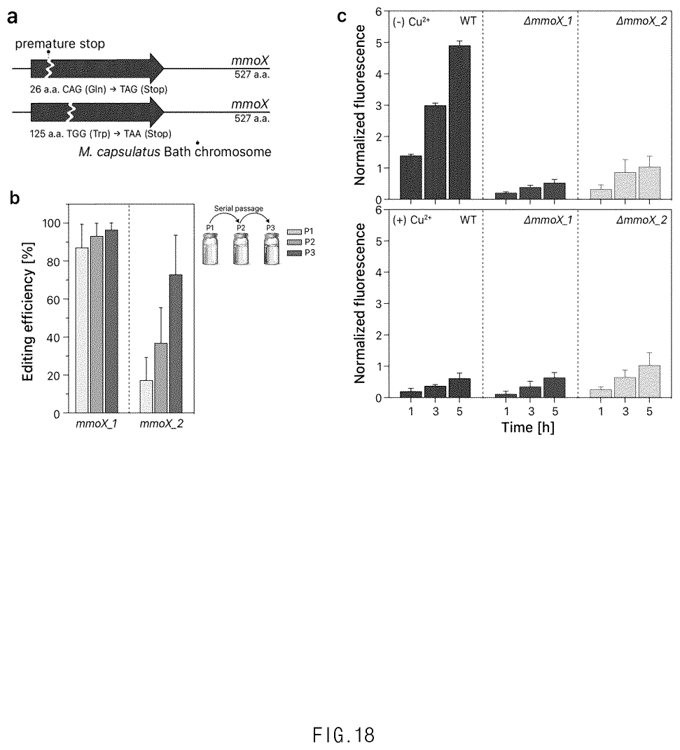

As a result, as shown in (b) in FIG. 18, it was confirmed that the ΔmmoX_1 strain, a mmoX knockout mutant, showed high editing efficiency (87%) from the initial passage (P1), whereas the ΔmmoX_2 strain, another mmoX knockout mutant obtained with mmoX 2 gRNA, showed the editing efficiency of up to 73% after the serial transfer, which was increased from 17.3% of the initial (P1). In addition, in order to examine whether the premature stop codon for the target gene is induced at a phenotypic level, the activities of sMMO against wild-type (WT) and mutant M. capsulatus Bath strain were determined by fluorescence-based coumarin oxidation assay by controlling the addition of copper ((c) in FIG. 18). The sMMO activities of the ΔmmoX_1 and ΔmmoX_2 mutant strains were 9.3 times and 4.7 times lower than that of the wild-type M. capsulatus Bath strain, respectively. As expected, the sMMO activity of the wild-type M. capsulatus Bath strain was much higher in the absence of copper than that in the presence of copper, whereas the sMMO activity of the mmoX knockout mutant strains were very low regardless of the addition of copper ((c) in FIG. 18).

Thus, the phenol-inducible CRISPR-BE system successfully generated an early stop codon in the sMMO gene in the M. capsulatus Bath, which was further verified by the genotype-phenotype relationship, showing that the target gene was knocked out.

9-3. Confirmation of Improvement in Isoprene Productivity

The increase of isoprene production was confirmed by comparing the ΔmmoX mutant strain generated using the phenol-inducible CRISPR-BE system of the disclosure with the wild-type strain. OD₆₀₀ was measured to determine the cell density, and productions of isoprene and organic acids were measured according to Experimental Examples 4-3 and 4-4 described above.

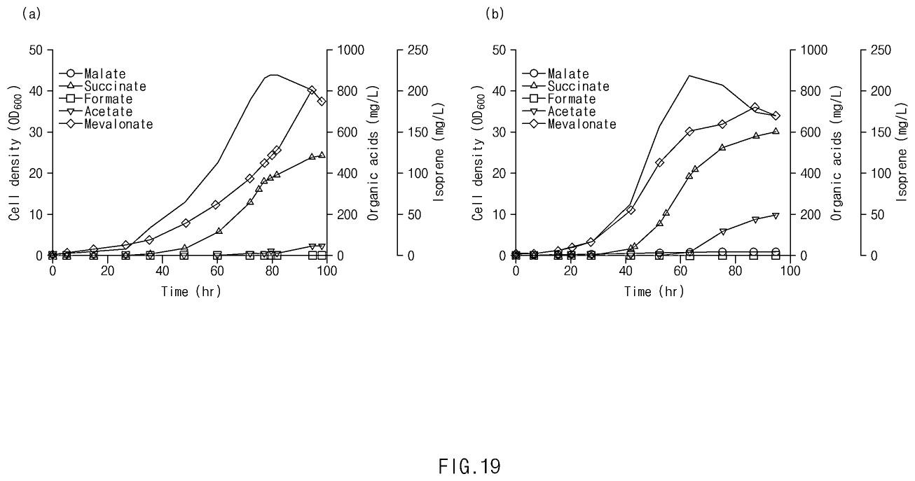

As a result of fermentation of the wild-type strain (Pₒ-PtIspS-MVA/Bath), OD₆₀₀ was reached 44.1 at its maximum, the isoprene production was 119.2 mg/L ((a) in FIG. 19), and as a result of fermentation of the ΔmmoX mutant strain (Pₒ-PtIspS-MVA/ΔmmoX), it was confirmed that OD₆₀₀ was reached 43.8 at its maximum and the isoprene production increased to 151.2 mg/L ((b) in FIG. 19). That is, high-density cell culture of the ΔmmoX mutant strain, which was a strain engineered to inactivate functional sMMO expression, increased isoprene production. As a result, despite exposure to the same amount of copper for the expression of pMMO, the wild-type strain (Pₒ-PtIspS-MVA/Bath) had a slower growth rate and lower isoprene production compared to the ΔmmoX mutant strain (Pₒ-PtIspS-MVA/ΔmmoX).

Through the above results, it was confirmed that the cytosine base editor using the Pₒ promoter operated well in methanotrophs, and it could be used to increase the productivity of desired substances by knocking out the target gene.

The preferred embodiments of the disclosure have been explained so far. A person skilled in the art will understand that the disclosure may be implemented in modifications without departing from the basic characteristics of the disclosure. Accordingly, the described embodiments should be considered in descriptive sense only and not for purposes of limitation. The scope of the disclosure is defined not by the detailed description herein but by the appended claims, and all differences within the scope will be construed as being included in the present inventive concept.

What is claimed is:

1. A vector for introduction into a methanotroph, comprising:
a first expression cassette comprising DmpR as a transcriptional regulator and a promoter operably linked thereto; and
a second expression cassette comprising a sequence encoding a target polypeptide and a Pₒ promoter operably linked thereto.

2. The vector of claim 1, wherein the target polypeptide is a marker, a reporter, a polypeptide capable of improving production of a desired metabolite, or a polypeptide capable of promoting growth of the methanotroph.

3. The vector of claim 1, wherein in the vector, expression of the target polypeptide is induced by a phenolic compound.

4. The vector of claim 1, wherein transcription directions of the first expression cassette and the second expression cassette are the same or opposite.

5. The vector of claim 1, wherein the vector is a plasmid.

6. The vector of claim 1, wherein the methanotroph is selected from the group consisting of Methylomonas sp., Methylobacter sp., Methylococcus sp., Methylosphaera sp., Methylocaldum sp., Methyloglobus sp., Methylosarcina sp., Methyloprofundus sp., Methylothermus sp., Methylohalobius sp., Methylogaea sp., Methylomarinum sp., Methylovulum sp., Methylomarinovum sp., Methylorubrum sp., Methyloparacoccus sp., Methylosinus sp., Methylocystis sp., Methylocella sp., Methylocapsa sp., Methylofurula sp., Methylacidiphilum sp., Methylacidimicrobium sp., and Methylomicrobium sp.

7. The vector of claim 1, wherein the methanotroph is Methylococcus capsulatus Bath or Methylosinus trichosporium OB3b.

8. A methanotroph in which the vector of claim 1 is introduced.


SEQUENCE LISTING (86 sequences)

SEQ ID NO: 1 - DNA, 1692 bp, synthetic construct
  misc_feature 1..1692
  source 1..1692: mol_type = other DNA
atgccgatcaagtacaagcctgaaatccagcactccgatttcaaggacctgaccaacctgatccacttccagagcacggaaggcaagatctggcttggcgaacaacgcatgctgttgctgcaggtttcagcaatggccagctttcgccgggaaatggtcaataccctgggcatcgaacgcgccaagggcttcttcctgcgccagggttaccagtccggcctgaaggatgccgaactggccaggaagcttagaccgaatgccagcgagtacgacatgttcctcgccggcccgcagctgcattcgctcaagggtctggtcaaggtccgccccaccgaggtcgatatcgacaaggaatgcgggcgcttctatgccgagatggagtggatcgactccttcgaggtggaaatctgccagaccgacctggggcagatgcaagacccggtgtgctggactctgctcggctacgcctgcgcctattcctcggcgttcatgggccgggaaatcatcttcaaggaagtcagctgccgcggctgcggcggcgacaagtgccgggtcattggcaagccggccgaagagtgggacgacgttgccagcttcaaacagtatttcaagaacgaccccatcatcgaggaactctacgagttgcaatcgcaactggtgtcgctgcgtaccaacctcgacaaacaggaaggccagtactacggcatcggtcagaccccggcctaccagaccgtgcgcaatatgatggacaaggccgcacagggcaaagtctcggtgctgctgcttggcgagaccggggtcggcaaggaggtcatcgcgcgtagcgtgcacctgcgcagcaaacgcgccgccgagccctttgtcgcggtgaactgtgcggcgatcccgccggacctgatcgagtccgaattgttcggcgtggaaaaaggcgccttcaccggcgccacccagtcacgcatgggccgcttcgagcgggccgacaagggcaccatcttccttgacgaggtgatcgaactcagcccgcgcgctcaggccagtctgctgcgcgtgctgcaagaaggcgagctggagcgagttggcgacaaccgcacgcgcaagatcgacgtaagggttatcgcagccacccacgaggacctggccgaagcggtcaaggccgggcgttttcgcgccgacctgtactaccggctgaacgttttcccggtggcgatcccggcgttgcgcgaacgccgcgaggacattccactgctggttgagcacttccttcagcgcttccaccaggagtacggcaagagaaccctcggcctttcagacaaagccctggaggcctgcctgcattacagttggccgggcaatatccgtgagctggagaacgtcatcgagcgcggcatcatcctcaccgatccgaacgaaagcatcagcgtgcaggcgctgttcccacgggcgccggaagagccgcagaccgccagcgagcgggtgtcgtcggacggcgtgctgattcagccaggcaatggccagggcagttggatcagccagttgttgagcagcggcctgagcctcgacgagatcgaggaaagcctgatgcgcaaagccatgcaacaggccaaccaaaacgtctccggtgccgcgcgcttgctcggcctaagccgaccggcactggcctatcggctgaagaaaatcggcatcgaaggctaa

SEQ ID NO: 2 - DNA, 1083 bp, synthetic construct
  source 1..1083: mol_type = other DNA
atgaacgaccccatcatcgaggaactctacgagttgcaatcgcaactggtgtcgctgcgtaccaacctcgacaaacaggaaggccagtactacggcatcggtcagaccccggcctaccagaccgtgcgcaatatgatggacaaggccgcacagggcaaagtctcggtgctgctgcttggcgagaccggggtcggcaaggaggtcatcgcgcgtagcgtgcacctgcgcagcaaacgcgccgccgagccctttgtcgcggtgaactgtgcggcgatcccgccggacctgatcgagtccgaattgttcggcgtggaaaaaggcgccttcaccggcgccacccagtcacgcatgggccgcttcgagcgggccgacaagggcaccatcttccttgacgaggtgatcgaactcagcccgcgcgctcaggccagtctgctgcgcgtgctgcaagaaggcgagctggagcgagttggcgacaaccgcacgcgcaagatcgacgtaagggttatcgcagccacccacgaggacctggccgaagcggtcaaggccgggcgttttcgcgccgacctgtactaccggctgaacgttttcccggtggcgatcccggcgttgcgcgaacgccgcgaggacattccactgctggttgagcacttccttcagcgcttccaccaggagtacggcaagagaaccctcggcctttcagacaaagccctggaggcctgcctgcattacagttggccgggcaatatccgtgagctggagaacgtcatcgagcgcggcatcatcctcaccgatccgaacgaaagcatcagcgtgcaggcgctgttcccacgggcgccggaagagccgcagaccgccagcgagcgggtgtcgtcggacggcgtgctgattcagccaggcaatggccagggcagttggatcagccagttgttgagcagcggcctgagcctcgacgagatcgaggaaagcctgatgcgcaaagccatgcaacaggccaaccaaaacgtctccggtgccgcgcgcttgctcggcctaagccgaccggcactggcctatcggctgaagaaaatcggcatcgaaggctaa

SEQ ID NO: 3 - DNA, 171 bp, synthetic construct
  source 1..171: mol_type = other DNA
tgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcg

SEQ ID NO: 4 - DNA, 23 bp, synthetic construct
  source 1..23: mol_type = other DNA
tttgtttaactttaagaaggaga

SEQ ID NO: 5 - DNA, 36 bp, synthetic construct
  source 1..36: mol_type = other DNA
tgcagggtcggcatcaatcattcttggaggagacac

SEQ ID NO: 6 - DNA, 36 bp, synthetic construct
  source 1..36: mol_type = other DNA
catcattcatagaatgtgttacggaggaaacaagta

SEQ ID NO: 7 - DNA, 36 bp, synthetic construct
  source 1..36: mol_type = other DNA
cgttcgatcaacctcaaaccaaaaaggaacatcgat

SEQ ID NO: 8 - DNA, 36 bp, synthetic construct
  source 1..36: mol_type = other DNA
tggacactttttcaacggcctgttaaggagaatgac

SEQ ID NO: 9 - DNA, 36 bp, synthetic construct
  source 1..36: mol_type = other DNA
gaaaataagaaaacgacaaatttggaggtaacttta

SEQ ID NO: 10 - DNA, 36 bp, synthetic construct
  source 1..36: mol_type = other DNA
gtgacccaacagcaagaactcgaaagaggagagatc

SEQ ID NO: 11 - DNA, 36 bp, synthetic construct
  source 1..36: mol_type = other DNA
ggcccctgtcaaccatcactttaggaggaacaaaca

SEQ ID NO: 12 - DNA, 36 bp, synthetic construct
  source 1..36: mol_type = other DNA
ccggtgttcacatcgatataacagttggaggtaaaa

SEQ ID NO: 13 - DNA, 36 bp, synthetic construct
  source 1..36: mol_type = other DNA
gtggcatcaatccaacaccaactcaggagaaatcag

SEQ ID NO: 14 - DNA, 702 bp, synthetic construct
  source 1..702: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

SEQ ID NO: 15 - DNA, 5756 bp, synthetic construct
  source 1..5756: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgctctgaaatgagctgttgacaattaatcatcggctcgtataatgtgtggaggtattcacacaggaaacagctatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagttgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 16 - DNA, 632 bp, synthetic construct
  source 1..632: mol_type = other DNA
gacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgct

SEQ ID NO: 17 - DNA, 589 bp, synthetic construct
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 18 - DNA, 110 bp, synthetic construct
  source 1..110: mol_type = other DNA
ccggccagcctcgcagagcaggattcccgttgagcaccgccaggtgcgaataagggacagtgaagaaggaacacccgctcgcgggtgggcctacttcacctatcctgccc

SEQ ID NO: 19 - DNA, 1149 bp, synthetic construct
  source 1..1149: mol_type = other DNA
ctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcat

SEQ ID NO: 20 - DNA, 372 bp, synthetic construct
  source 1..372: mol_type = other DNA
tcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccat

SEQ ID NO: 21 - DNA, 117 bp, synthetic construct
  source 1..117: mol_type = other DNA
atggctgatgaaaccaagccaaccaggaagggcagcccacctatcaaggtgtactgccttccagacgaacgaagagcgattgaggaaaaggcggcggcggccggcatgagcctgtag

SEQ ID NO: 22 - DNA, 816 bp, synthetic construct
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 23 - DNA, 20 bp, synthetic construct
  source 1..20: mol_type = other DNA
agccgtgtgcgagacaccgc

SEQ ID NO: 24 - DNA, 33 bp, synthetic construct
  source 1..33: mol_type = other DNA
cccgacaatctagatagctgtttcctgtgtgaa

SEQ ID NO: 25 - DNA, 32 bp, synthetic construct
  source 1..32: mol_type = other DNA
cagctatctagattgtcgggaagatgcgtgat

SEQ ID NO: 26 - DNA, 20 bp, synthetic construct
  source 1..20: mol_type = other DNA
agtctggaaagaaatgcata

SEQ ID NO: 27 - DNA, 33 bp, synthetic construct
  source 1..33: mol_type = other DNA
ttcagagactagtttcgcaacctagtgaatgag

SEQ ID NO: 28 - DNA, 33 bp, synthetic construct
  source 1..33: mol_type = other DNA
ttgcgaaactagtctctgaaatgagctgttgac

SEQ ID NO: 29 - DNA, 20 bp, synthetic construct
  source 1..20: mol_type = other DNA
tcacgcatcttcccgacaat

SEQ ID NO: 30 - DNA, 40 bp, synthetic construct
  source 1..40: mol_type = other DNA
tcattcactaggttgcgaaatcagagattttgagacacaa

SEQ ID NO: 31 - DNA, 30 bp, synthetic construct
  source 1..30: mol_type = other DNA
tgctcaccatatgtatatctccttcttaaa

SEQ ID NO: 32 - DNA, 30 bp, synthetic construct
  source 1..30: mol_type = other DNA
agatatacatatggtgagcaagggcgagga

SEQ ID NO: 33 - DNA, 40 bp, synthetic construct
  source 1..40: mol_type = other DNA
tcacgcatcttcccgacaatctacttgtacagctcgtcca

SEQ ID NO: 34 - DNA, 45 bp, synthetic construct
  source 1..45: mol_type = other DNA
ctatcaacaggagtccaagactagtttgacaccatcgaatggtgc

SEQ ID NO: 35 - DNA, 45 bp, synthetic construct
  source 1..45: mol_type = other DNA
aaagttaaacaaaattatttctagagtgtgaaattgttatccgct

SEQ ID NO: 36 - DNA, 45 bp, synthetic construct
  source 1..45: mol_type = other DNA
ctatcaacaggagtccaagactagtacgttcgtaatcaagccact

SEQ ID NO: 37 - DNA, 45 bp, synthetic construct
  source 1..45: mol_type = other DNA
aaagttaaacaaaattatttctagacatggtcatgactccattat

SEQ ID NO: 38 - DNA, 45 bp, synthetic construct
  source 1..45: mol_type = other DNA
ctatcaacaggagtccaagactagtagcggataacaatttcacac

SEQ ID NO: 39 - DNA, 45 bp, synthetic construct
  source 1..45: mol_type = other DNA
aaagttaaacaaaattatttctagatctccaggttggcggattgc

SEQ ID NO: 40 - DNA, 9154 bp, synthetic construct
  source 1..9154: mol_type = other DNA
aggcatcaaataaaacgaaaggctcagtcgaaagactgggcctttcgttttatctgttgtttgtcggtgaacgctctcctgagtaggacaaatccgccgccctagacagctgggcgcgccgtagaaaagatcaaaggatcttcttgagatcctttttttctgcgggggatcaggaccgctgccggagcgcaacccactcactacagcagagccatgtagggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcgacggatcgatccggggaattaattccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatagtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaagccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccacctgcatcgcggccggcctacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaaggacaacgcgcggaccgcggtccaattaattattagaaaaattcatccagcatcagatgaaattgcagtttgttcatatccggattatcaatgccatatttctgaaacagacgtttttgcaggctcgggctaaattcgcccaggcagttccacagaatggccagatcctgataacgatccgcaatgcccacacggcccacatcaatgcagccaatcagtttgccttcatcgaaaatcaggttatccaggctaaaatcgccgtgggtcaccacgctatccgggctaaacggcagcagtttatgcatttctttccacacctgttccaccggccagccgttacgttcatcatcaaaatcgctcgcatccaccaggccgttgttcatacggctctgcgcctgggccagacgaaacacacgatcgctgttaaacgggcagttgcacaccggaatgctatgcagacgacgcagaaacacggccagcgcatccacaatgttttcgccgctatccggatattcttccagcacctgaaacgcggttttgcccggaatcgcggtggtcagcagccacgcatcatccggggtgcgaataaaatgtttaatggtcggcagcggcataaattcggtcagccagttcagacgcaccatttcatcggtcacatcgttcgccacgctgcctttgccatgtttcagaaacagttccggcgcatccggtttgccatacagacgataaatggtcgcgccgctctgacccacgttatcacgcgcccatttatagccatacagatccgcatccatgttgctgttcagacgcggacggctacagctcgtttcacgctgaatatggctcataacaccccttgtattactgtttatgtaagcagacagttttattgttcatgatgatatatttttatcttgtgcaatgtaacatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtcgccagggttttcccagtcattaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagtctagaaataattttgtttaactttaagaaggagatatacatatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggatatattccgcttcctcggcgaccggggcggccgcctcgaggaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggcgcccaatacgcaaaccgtgaaattgttatccgctttaattaa

SEQ ID NO: 41 - DNA, 5818 bp, synthetic construct
  source 1..5818: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtcgccagggttttcccagtcattaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagtctagaaataattttgtttaactttaagaaggagatatacatatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 42 - DNA, 6553 bp, synthetic construct
  source 1..6553: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacactctagaaataattttgtttaactttaagaaggagatatacatatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 43 - DNA, 7084 bp, synthetic construct
  source 1..7084: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtacgttcgtaatcaagccacttcctttttgcattgacgcagggtgtcggaaggcaactcgccgaacgcgctcctatagttttcagcgaagcgtcccaaatgtaagaagccgtagtctagggctatctcagttatactacgcacattggcactgggatcgttcaagcaggcgcggatgctttcgagcttgcggttgcggatgtagttcttcggcgtggtgccggcgtgcttctcgaacaaattgtagagcgagcgtggactcatcatcgccagctccgctaaccgctcaaggctgatattccgtttgagattctcctcaatgaattgaacgactcgctcgaaagacgggttacctttgctgaaaatttcacggctgacattgctgcccagcatttcgagcagcttggaagcgatgatccccgcatagtgctcttggacccgaggcatcgactttgtatgttccgcttcgtcacaaactaacccgagtagattgataaagccatcgagttgctggagattgtgtcgcgcggcgaaacggataccctccctcggcttgtgccaattgttgtcactgcacgcccgatcaaggaccactgagggcaatttaacgataaatttctcgcaatcttctgaataggtcaggtcggcttggtcatccggattgagcagcaatagttcgcccggcgcaaaatagtgctcctggccatggccacgccacaggcaatggcctttgagtattatttgcagatgataacaggtctctaatccaggcgagattaccctcacgctaccgccgtagctgattcgacacagatcgaggcatccgaagattctgtggtgcagcctgcctgccgggcgcccgcccttgggcaggcgaatagagtgcgtaccgacatactggttaacataatcggagactgcatagggctcggcgtggacgaagatctgacttttctcgttcaataagcaaaaatccatagttcacggttctcttattttaatgtgggctgcttggtgtgatgtagaaaggcgccaagtcgatgaaaatgcatctcgacgtgatgcgtatacgggttacccccattgccacgttgcgccatcctttttgcaatcagtgaccacttttccaagcaaaaataacgccaagcagaacgaagacgttctttttaagaagcgagaacaccagaagttcgtgctgtcggggcatggggcgacgaattggcggataaaggggatctgctggatattacggcctttttaaagaccgtaaagaaaaataagcacaagttttatccggcctttattcacattcttgcccgcctgatgaatgctcatccgtaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggaattagcttgatctgaccaacgaccggtagcggagctatccaacggcggtataccaggaaaacacacagcaggtacatcagaacagtaccatgactgaagaacaaatagttttttcctgatccataaagcagaacggcctgctccatgacaaatctggctccccaactaatgccccatgcagccagcataaccagcataaagtgcagtgtccggtttgatagggataagtccagccttgcaagaagcggatacaggagtgcaaaaaatggctatctctagtaaggcctaccccttaggctttatgcaacagaaacaataataatggagtcatgaccatgtctagaaataattttgtttaactttaagaaggagatatacatatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 44 - DNA, 5984 bp, synthetic construct
  source 1..5984: mol_type = other DNA
ttaattaaagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagaaggagatatacatatgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaacgggaccacatggttctgcacgaatatgtgaatgcggcaggtattacgtggagccatccgcagttcgaaaaataagatcctacaaggaggtaccagtcgtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaattagtagcccgcctaatgagcgggcttttttttaattcccctatttgtttatttttctaaatacattcaaatatgtatccgctcatgagacaataaccctgataaatgcttcaataatattgaaaaaggaagagtatgagcattcagcattttcgtgtggcgctgattccgttttttgcggcgttttgcctgccggtgtttgcgcatccggaaaccctggtgaaagtgaaagatgcggaagatcaactgggtgcgcgcgtgggctatattgaactggatctgaacagcggcaaaattctggaatcttttcgtccggaagaacgttttccgatgatgagcacctttaaagtgctgctgtgcggtgcggttctgagccgtgtggatgcgggccaggaacaactgggccgtcgtattcattatagccagaacgatctggtggaatatagcccggtgaccgaaaaacatctgaccgatggcatgaccgtgcgtgaactgtgcagcgcggcgattaccatgagcgataacaccgcggcgaacctgctgctgacgaccattggcggtccgaaagaactgaccgcgtttctgcataacatgggcgatcatgtgacccgtctggatcgttgggaaccggaactgaacgaagcgattccgaacgatgaacgtgataccaccatgccggcagcaatggcgaccaccctgcgtaaactgctgacgggtgagctgctgaccctggcaagccgccagcaactgattgattggatggaagcggataaagtggcgggtccgctgctgcgtagcgcgctgccggctggctggtttattgcggataaaagcggtgcgggcgaacgtggcagccgtggcattattgcggcgctgggcccggatggtaaaccgagccgtattgtggtgatttataccaccggcagccaggcgacgatggatgaacgtaaccgtcagattgcggaaattggcgcgagcctgattaaacattggtaaaccgatacaattaaaggctccttttggagcctttttttttggacgacccttgtccttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgtaggccggccctaccggcgcggcagcgttacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggggggcgcgcccagctgtctagggcggcggatttgtcctactcaggagagcgttcaccgacaaacaacagataaaacgaaaggcccagtctttcgactgagcctttcgttttatttgatgcct

SEQ ID NO: 45 - DNA, 2172 bp, synthetic construct
  source 1..2172: mol_type = other DNA
ctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaaataattttgtttaacttt

SEQ ID NO: 46 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaaataattttgtttaactttaagaaggagatatacatatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 47 - protein, 560 aa, synthetic construct
MPIKYKPEIQHSDFKDLTNLIHFQSTEGKIWLGEQRMLLLQVSAMASFRREMVNTLGIERAKGFFLRQGYQSGLKDAELARKLRPNASEYDMFLAGPQLHSLKGLVKVRPTEVDIDKECGRFYAEMEWIDSFEVEICQTDLGQMQDPVCWTLLGYACAYSSAFMGREIIFKEVSCRGCGGDKCRVIGKPAEEWDDVASFKQYFKNDPIIEELYELQSQLVSLRTNLDKQEGQYYGIGQTPAYQTVRNMMDKAAQGKVSVLLLGETGVGKEVIARSVHLRSKRAAEPFVAVNCAAIPPDLIESELFGVEKGAFTGATQSRMGRFERADKGTIFLDEVIELSPRAQASLLRVLQEGELERVGDNRTRKIDVRVIAATHEDLAEAVKAGRFRADLYYRLNVFPVAIPALRERREDIPLLVEHFLQRFHQEYGKRTLGLSDKALEACLHYSWPGNIRELENVIERGIILTDPNESISVQALFPRAPEEPQTASERVSSDGVLIQPGNGQGSWISQLLSSGLSLDEIEESLMRKAMQQANQNVSGAARLLGLSRPALAYRLKKIG

SEQ ID NO: 48 - DNA, 6610 bp, synthetic construct
  source 1..6610: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaaataattttgtttaactttaagaaggagatatacatatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 49 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagatgcagggtcggcatcaatcattcttggaggagacacatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 50 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagacatcattcatagaatgtgttacggaggaaacaagtaatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 51 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagacgttcgatcaacctcaaaccaaaaaggaacatcgatatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 52 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagatggacactttttcaacggcctgttaaggagaatgacatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 53 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagagaaaataagaaaacgacaaatttggaggtaactttaatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 54 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagagtgacccaacagcaagaactcgaaagaggagagatcatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 55 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaggcccctgtcaaccatcactttaggaggaacaaacaatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 56 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaccggtgttcacatcgatataacagttggaggtaaaaatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 57 - DNA, 7219 bp, synthetic construct
  source 1..7219: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaatcagagattttgagacacaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcagactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagagtggcatcaatccaacaccaactcaggagaaatcagatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 58 - DNA, 11948 bp, synthetic construct
  source 1..11948: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcaaactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaaataattttgtttaactttaagaaggagatatacatatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctaactgctgccaccgctgagcaataactcgagctgtcaccggatgtgctttccggtctgatgagtccgtgaggacgaaacagcctctacaaataattttgtttaagtttccagccgaggccgagcgttttagagctagaaatagcaagttaaaataaggctagtccgttatcaacttgaaaaagtggcaccgagtcggtgctttttttgacccggggcggccgcctcgattaattaaaggcatcaaataaaacgaaaggctcagtcgaaagactgggcctttcgttttatctgttgtttgtcggtgaacgctctcctgagtaggacaaatccgccgccctagacagctgggcgcgccattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 59 - DNA, 12000 bp, synthetic construct
  source 1..12000: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgacccgctagggacgtgaagtcgattccttcgatggttagcaatcaaagactcagagtgctgtctggagcgtgaatctaacggtacgtatctcgattgctcggtcgctattcgcactctgcgaaagttcgtaccgctcattcactaggttgcgaaaatttaaatcgtaattattggggacccctggattctcaccaataaaaaacgcccggcggcaaccgagcgttctgaacaaatccagatggagttctgaggtcattactggatctatcaacaggagtccaagactagtagcggataacaatttcacacaggattagccttcgatgccgattttcttcagccgataggccagtgccggtcggcttaggccgagcaagcgcgcggcaccggagacgttttggttggcctgttgcatggctttgcgcatcaggctttcctcgatctcgtcgaggctcaggccgctgctcaacaactggctgatccaactgccctggccattgcctggctgaatcagcacgccgtccgacgacacccgctcgctggcggtctgcggctcttccggcgcccgtgggaacagcgcctgcacgctgatgctttcgttcggatcggtgaggatgatgccgcgctcgatgacgttctccagctcacggatattgcccggccaactgtaatgcaggcaggcctccagggctttgtctgaaaggccgagggttctcttgccgtactcctggtggaagcgctgaaggaagtgctcaaccagcagtggaatgtcctcgcggcgttcgcgcaacgccgggatcgccaccgggaaaacgttcagccggtagtacaggtcggcgcgaaaacgcccggccttgaccgcttcggccaggtcctcgtgggtggctgcgataacccttacgtcgatcttgcgcgtgcggttgtcgccaactcgctccagctcgccttcttgcagcacgcgcagcaaactggcctgagcgcgcgggctgagttcgatcacctcgtcaaggaagatggtgcccttgtcggcccgctcgaagcggcccatgcgtgactgggtggcgccggtgaaggcgcctttttccacgccgaacaattcggactcgatcaggtccggcgggatcgccgcacagttcaccgcgacaaagggctcggcggcgcgtttgctgcgcaggtgcacgctacgcgcgatgacctccttgccgaccccggtctcgccaagcagcagcaccgagactttgccctgtgcggccttgtccatcatattgcgcacggtctggtaggccggggtctgaccgatgccgtagtactggccttcctgtttgtcgaggttggtacgcagcgacaccagttgcgattgcaactcgtagagttcctcgatgatggggtcgttcttgaaatactgtttgaagctggcaacgtcgtcccactcttcggccggcttgccaatgacccggcacttgtcgccgccgcagccgcggcagctgacttccttgaagatgatttcccggcccatgaacgccgaggaataggcgcaggcgtagccgagcagagtccagcacaccgggtcttgcatctgccccaggtcggtctggcagatttccacctcgaaggagtcgatccactccatctcggcatagaagcgcccgcattccttgtcgatatcgacctcggtggggcggaccttgaccagacccttgagcgaatgcagctgcgggccggcgaggaacatgtcgtactcgctggcattcggtctaagcttcctggccagttcggcatccttcaggccggactggtaaccctggcgcaggaagaagcccttggcgcgttcgatgcccagggtattgaccatttcccggcgaaagctggccattgctgaaacctgcagcaacagcatgcgttgttcgccaagccagatcttgccttccgtgctctggaagtggatcaggttggtcaggtccttgaaatcggagtgctggatttcaggcttgtacttgatcggcatgtaagcgaggcccctatttatttttagatggggaaatcagggtcgccgctatagcgcaaggcaggcggcgattccagatggggtcatggggaaaatcggcagttttttcacctgcgccagctccccatagccgacctcagtatctggcaaaagtcaaccaaatgatcaatcgacggcagtgactttagtgctagagatcagccttcagctgcgccatgagcatttgctcaagcggccttgggcaattgatcaaatgcttaaaaagcctgcgcaagcgcggcttaatttcgctcgctccgatcattctaaaaattagaaacacattgaaaaacaataccttgaagtcggttttcagaccttggcacagctgttgcactttgtcctgcgcaatccgccaacctggagatctagaaataattttgtttaactttaagaaggagatatacatatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctaactgctgccaccgctgagcaaagctgctctgaaatgagctgttgacaattaatcatcggctcgtataatgtgtggactgcagctgtcaccggatgtgctttccggtctgatgagtccgtgaggacgaaacagcctctacaaataattttgtttaagtttccagccgaggccgagcgttttagagctagaaatagcaagttaaaataaggctagtccgttatcaacttgaaaaagtggcaccgagtcggtgctttttttgacccggggcggccgcctcgattaattaaaggcatcaaataaaacgaaaggctcagtcgaaagactgggcctttcgttttatctgttgtttgtcggtgaacgctctcctgagtaggacaaatccgccgccctagacagctgggcgcgccattgtcgggaagatgcgtgatctgatccttccactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 60 - DNA, 30 bp, synthetic construct
  source 1..30: mol_type = other DNA
cgcttacatgaacgaccccatcatcgagga

SEQ ID NO: 61 - DNA, 30 bp, synthetic construct
  source 1..30: mol_type = other DNA
tggggtcgttcatgtaagcgaggcccctat

SEQ ID NO: 62 - DNA, 20 bp, synthetic construct
  source 1..20: mol_type = other DNA
ggccattgctgaaacctgca

SEQ ID NO: 63 - DNA, 20 bp, synthetic construct
  source 1..20: mol_type = other DNA
tctagatctccaggttggcg

SEQ ID NO: 64 - DNA, 20 bp, synthetic construct
  source 1..20: mol_type = other DNA
gatcagcgtgccgtcctgca

SEQ ID NO: 65 - DNA, 76 bp, synthetic construct
  source 1..76: mol_type = other DNA
cgccaacctggagatctagatgcagggtcggcatcaatcattcttggaggagacacatggtgagcaagggcgagga

SEQ ID NO: 66 - DNA, 76 bp, synthetic construct
  source 1..76: mol_type = other DNA
cgccaacctggagatctagacatcattcatagaatgtgttacggaggaaacaagtaatggtgagcaagggcgagga

SEQ ID NO: 67 - DNA, 76 bp, synthetic construct
  source 1..76: mol_type = other DNA
cgccaacctggagatctagacgttcgatcaacctcaaaccaaaaaggaacatcgatatggtgagcaagggcgagga

SEQ ID NO: 68 - DNA, 76 bp, synthetic construct
  source 1..76: mol_type = other DNA
cgccaacctggagatctagatggacactttttcaacggcctgttaaggagaatgacatggtgagcaagggcgagga

SEQ ID NO: 69 - DNA, 76 bp, synthetic construct
  source 1..76: mol_type = other DNA
cgccaacctggagatctagagaaaataagaaaacgacaaatttggaggtaactttaatggtgagcaagggcgagga

SEQ ID NO: 70 - DNA, 76 bp, synthetic construct
  source 1..76: mol_type = other DNA
cgccaacctggagatctagagtgacccaacagcaagaactcgaaagaggagagatcatggtgagcaagggcgagga

SEQ ID NO: 71 - DNA, 76 bp, synthetic construct
  source 1..76: mol_type = other DNA
cgccaacctggagatctagaggcccctgtcaaccatcactttaggaggaacaaacaatggtgagcaagggcgagga

SEQ ID NO: 72 - DNA, 76 bp, synthetic construct
  source 1..76: mol_type = other DNA
cgccaacctggagatctagaccggtgttcacatcgatataacagttggaggtaaaaatggtgagcaagggcgagga

SEQ ID NO: 73 - DNA, 76 bp, synthetic construct
  source 1..76: mol_type = other DNA
cgccaacctggagatctagagtggcatcaatccaacaccaactcaggagaaatcagatggtgagcaagggcgagga

SEQ ID NO: 74 - DNA, 20 bp, synthetic construct
  source 1..20: mol_type = other DNA
ggcacgatccctgtaactag

SEQ ID NO: 75 - DNA, 19 bp, synthetic construct
  source 1..19: mol_type = other DNA
aatgaagccgtcggaaaac

SEQ ID NO: 76 - DNA, 19 bp, synthetic construct
  source 1..19: mol_type = other DNA
gcttcatgcccttccacag

SEQ ID NO: 77 - DNA, 24 bp, synthetic construct
  source 1..24: mol_type = other DNA
agatattattgaaaaggagaccgg

SEQ ID NO: 78 - DNA, 30 bp, synthetic construct
  source 1..30: mol_type = other DNA
ttttatttgatgcctttaattaatcgaggc

SEQ ID NO: 79 - DNA, 32 bp, synthetic construct
  source 1..32: mol_type = other DNA
ttctacaaactcttattgtcgggaagatgcgt

SEQ ID NO: 80 - DNA, 44 bp, synthetic construct
  source 1..44: mol_type = other DNA
ggatacagagcaagccatatgtatatctccttcttaaagttaaa

SEQ ID NO: 81 - DNA, 24 bp, synthetic construct
  source 1..24: mol_type = other DNA
agatatacatatggcttgctctgt

SEQ ID NO: 82 - DNA, 29 bp, synthetic construct
  source 1..29: mol_type = other DNA
ttcctgtgtgaattgcgttgcgctctaga

SEQ ID NO: 83 - DNA, 25 bp, synthetic construct
  source 1..25: mol_type = other DNA
caacgcaattcacacaggaaacagc

SEQ ID NO: 84 - DNA, 30 bp, synthetic construct
  source 1..30: mol_type = other DNA
atcttcccgacaataagagtttgtagaaac

SEQ ID NO: 85 - DNA, 20 bp, synthetic construct
  source 1..20: mol_type = other DNA
atgcacaggaagtgcaccgt

SEQ ID NO: 86 - DNA, 20 bp, synthetic construct
  source 1..20: mol_type = other DNA
atcccacagcatcccggtag